(19) United States
(12) Patent Application Publication
(10) Pub. No.: US 2008/0219943 A1
(43) Pub. Date: Sep. 11, 2008

(54) Composition for Coating Keratin Fibres Comprising a Block Polymer and a Semicrystalline Polymer

(76) Inventor: Valerie De La Poterie, Lailly-en-Val (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/585,818

(22) PCT Filed: Jan. 11, 2005
(86) PCT No.: PCT/IB05/00236

§ 371 Date: Jul. 12, 2006

(30) Foreign Application Priority Data
Jan. 13, 2004 (FR) 0450077

(51) Int. Cl. A61K 8/72; A61Q 1/10

(52) U.S. Cl. 424/70.7; 424/70.11; 424/70.16; 424/70.12

(57) ABSTRACT

A subject of the invention is a composition for coating keratin fibres comprising a cosmetically acceptable medium, a block polymer, and at least one semicrystalline polymer. A subject of the invention is also the use of such a composition for obtaining a makeup for the keratin fibres, in particular the eyelashes, which is charging and/or homogeneous and/or smooth and/or has good staying power.

The subject of the present invention is a cosmetic composition for coating keratin fibres comprising a block polymer and a semicrystalline polymer.

The invention also relates to a cosmetic process for making up or treating keratin fibres such as the eyelashes, the eyebrows and the hair.

The composition according to the invention may be a makeup composition, also called mascara, a makeup base for keratin fibres or base coat, a composition to be applied over makeup, also known as top coat, or a composition for treating keratin fibres. More especially, the composition according to the invention is a mascara.

The term “mascara” is understood as meaning a composition intended to be applied to the eyelashes: it may be a makeup composition for the eyelashes, a makeup base for the eyelashes, a composition to be applied over a mascara, also known as top coat, or a cosmetic treatment composition for the eyelashes. The mascara is more particularly intended for the eyelashes of human beings, but also for false eyelashes.

Preferably, the composition according to the invention is a leave-in composition.

Makeup compositions for the eyes, and in particular for the eyelashes, such as mascaras, may be provided in various forms: for example in the form of biphasic oil-in-water or O/W or water-in-oil W/O emulsions, or of aqueous or anhydrous dispersions.

It is generally through the qualitative and quantitative choice of the waxes and polymers that the desired specificities of application are adjusted for the makeup compositions, such as their fluidity, their covering power and/or their curling power. Thus, it is possible to prepare various compositions which, when applied in particular to the eyelashes, induce varied effects of the lengthening, curling and/or thickening type (charging or volumizing effect).

It is known from the prior art that the higher the content of solids (provided in part by a fatty phase consisting, for example, of one or more waxes or of one or more lipophilic polymers) in a composition, the greater the deposition of material on the eyelashes and therefore the more the result obtained will be volumizing.

However, the increase in the content of solids in a composition, such as an emulsion or dispersion, causes an increase in the consistency of the product obtained and therefore a delicate and difficult application to the eyelashes because the product is thick and viscous, it forms a deposit with difficulty, in a heterogeneous manner and in packets. The increase in the content of solids is therefore often limited by the increase in consistency and does not exceed 45% of the total weight of the composition. This limitation on the content of solids is often linked to the impossibility of increasing, on the one hand, the wax content in the fatty phase which does not exceed 25% for reasons of feasibility (the compositions comprising between 20 and 25% by weight of wax are often very thick, compact, difficult to apply and have unsatisfactory cosmetic properties) and, on the other hand, of incorporating fat-soluble polymers in a large amount, which considerably increases the viscosity of the composition.

Another means of increasing the content of solids is to incorporate solid particles such as fillers or pigments, but the increase in consistency also limits the maximum percentage of solids; furthermore, the use of solid particles in a large quantity does not promote homogeneous and smooth deposition not only because of the consistency but also because of the size of the particles introduced, which gives a granular and unsmooth appearance to the deposit.

That is generally the case for the so-called volumizing mascaras which are difficult to apply and which give a heterogeneous makeup.

It is therefore difficult to obtain a makeup composition for the keratin fibres, comprising a high content of solids and therefore a satisfactory volumizing effect, having an easy and homogeneous application.

Moreover, the increase in the solids content and the inhomogeneity of the deposit causes a less satisfactory staying power of the composition film: the latter is not sufficiently resistant to rubbing, in particular with the fingers, and/or to water, during bathing or showers for example, or to tears, to sebum or to sweat. The mascara tends in this case to crumble over time: grains form a deposit and leave marks around the eyes. The crumbling of the film causes a substantial loss of the intensity of the colour of the makeup, thus forcing the consumer to repeat the application of the mascara.

The aim of the present invention is therefore to propose another route for formulating a composition for coating the keratin fibres leading to a keratin fibre charging effect, and which completely or partially solves the problems linked to conventional routes of formulation. In addition, the compositions according to the invention allow smooth and homogeneous application and lead to a makeup on the keratin fibres having good resistance in particular to water and/or to sweat and/or to sebum.

The inventors have discovered that such a composition could be obtained by using a particular block polymer and a semicrystalline polymer.

Surprisingly, the incorporation of such a block polymer at high or very high contents (which may be up to 50% by weight) makes it possible to significantly increase the dry matter content of a composition for coating keratin fibres, while preserving a consistency which allows easy application to the keratin fibres. In addition, the combination of such a particular block polymer with a semicrystalline polymer leads, after application to the keratin fibres, to a makeup film with good staying power over time: the film does not crumble and has good resistance in particular to water, to sweat or to sebum.

More precisely, a subject of the invention is a composition for coating keratin fibres comprising a cosmetically acceptable organic liquid medium, at least one film-forming linear ethylenic block polymer, called in the text that follows “block polymer” and at least one semicrystalline polymer, different from the film-forming ethylenic block polymer.

A subject of the invention is also a cosmetic process for making up or for the nontherapeutic care of keratin fibres, in particular the eyelashes, comprising the application of a composition as defined above to the keratin fibres.

A subject of the invention is also the use of a composition as defined above for obtaining a makeup for the keratin fibres, in particular the eyelashes, which is charging and/or has good resistance in particular to water and/or to sweat and/or to sebum.

A subject of the invention is also the use of the combination of a film-forming linear ethylenic block polymer, and a semicrystalline polymer in a composition for coating keratin fibres, in order to obtain a composition that is easy to apply to the keratin fibres and/or leading to a makeup that is charging and/or has good resistance in particular to water and/or to sweat and/or to sebum on the said keratin fibres.

The term “cosmetically acceptable” organic liquid medium means an organic liquid medium that is compatible with the eyelashes or the skin.

### 1) Block Polymer

The polymer of the composition according to the invention is a film-forming linear ethylenic block polymer.

The term “ethylenic” polymer means a polymer obtained by polymerizing monomers comprising an ethylenic unsaturation.

The term “block” polymer means a polymer comprising at least 2 different blocks, preferably at least 3 different blocks.

The polymer is a polymer with a linear structure. In contrast, a polymer of non-linear structure is, for example, a polymer of branched, starburst or grafted structure, or the like.

The term “film-forming” polymer means a polymer capable of forming, by itself or in the presence of an auxiliary film-forming agent, a continuous film that adheres to a support and especially to keratin materials.

Advantageously, the block polymer of the composition according to the invention is free of styrene. The term “polymer free of styrene” means a polymer containing less that 10% by weight, relative to the total weight of the polymer, preferably less than 5% by weight, even better less than 2% by weight, even better less than 1% by weight, or not even containing none of a styrene monomer such as styrene, styrene derivatives such as methylstyrene, chlorostyrene or chloromethylstyrene of styrene or of styrene derivatives such as for example methylstyrene, chlorostyrene or chloromethylstyrene.

According to one embodiment, the block polymer of the inventive composition is derived from aliphatic ethylenic monomers. The term “aliphatic monomer” means a monomer comprising no aromatic groups.

According to one embodiment, the block polymer is an ethylenic polymer derived from aliphatic ethylenic monomers comprising a carbon-carbon double bond and at least one ester group —COO— or amide group —CON—. The ester group may be linked to one of the two unsaturated carbons via the carbon atom or the oxygen atom. The amide group may be linked to one of the two unsaturated carbons via the carbon atom or the nitrogen atom.

Preferably, the block polymer of the composition according to the invention comprises at least one first block and at least one second block having different glass transition temperatures (Tg), the said first and second blocks being linked together via an intermediate block comprising at least one constituent monomer of the first block and at least one constituent monomer of the second block.

The term “at least one” block means one or more blocks.

It is pointed out that, in the text hereinabove and hereinbelow, the terms “first” and “second” blocks do not in any way condition the order of the said blocks in the polymer structure.

Advantageously, the first and second blocks of the block polymer are mutually incompatible.

The term “mutually incompatible blocks” means that the mixture formed from the polymer corresponding to the first block and from the polymer corresponding to the second block is not miscible in the organic liquid medium that is contained in major amount by weight in the organic liquid medium of the composition, at room temperature (25° C.) and atmospheric pressure (10⁵ Pa), for a content of the polymer mixture of greater than or equal to 5% by weight, relative to the total weight of the mixture (polymers and solvent), it being understood that:

i) the said polymers are present in the mixture in a content such that the respective weight ratio ranges from 10/90 to 90/10, and
ii) each of the polymers corresponding to the first and second blocks has an average (weight-average or number-average) molecular mass equal to that of the block polymer ±15%.

When the organic liquid medium comprises a mixture of organic liquids, in the case of two or more liquids present in identical mass proportions, the said polymer mixture is immiscible in at least one of them.

Obviously, when the organic liquid medium comprises only one organic liquid, the latter is the major organic liquid.

Advantageously, the major organic liquid of the composition is the organic solvent for polymerizing the block polymer or the major organic solvent of the mixture of organic solvents for polymerizing the block polymer. The intermediate block is a block comprising at least one constituent monomer of the first block and at least one constituent monomer of the second block of the polymer makes it possible to “compatibilize” these blocks.

Preferably, the block copolymer of the invention is present in the organic liquid medium of the composition.

Preferably, the block polymer comprises no silicon atoms in its skeleton. The term “skeleton” means the main chain of the polymer, as opposed to the pendant side chains.

Preferably, the block polymer is not water-soluble, i.e. the polymer is not soluble in water or in a mixture of water and linear or branched lower monoalcohols containing from 2 to 5 carbon atoms, for instance ethanol, isopropanol or n-propanol, without pH modification, at an active material content of at least 1% by weight, at room temperature (25° C.).

Preferably, the polymer according to the invention is not an elastomer.

The term “non-elastomeric polymer” means a polymer which, when it is subjected to a constraint intended to stretch it (for example by 30% relative to its initial length), does not return to a length substantially identical to its initial length when the constraint ceases.

More specifically, the term “non-elastomeric polymer” denotes a polymer with an instantaneous recovery Rᵢ<50% and a delayed recovery R₂ₕ<70% after having been subjected to a 30% elongation. Preferably, Rᵢ is <30% and R₂ₕ<50%.

More specifically, the non-elastomeric nature of the polymer is determined according to the following protocol:

A polymer film is prepared by pouring a solution of the polymer into a Teflon-coated mould, followed by drying for 7 days in an environment conditioned at 23±5° C. and 50±10% relative humidity.

A film about 100 μm thick is thus obtained, from which are cut rectangular specimens (for example using a punch) 15 mm wide and 80 mm long.

This sample is subjected to a tensile stress using a machine sold under the reference Zwick, under the same temperature and humidity conditions as for the drying.

The specimens are pulled at a speed of 50 mm/min and the distance between the jaws is 50 mm, which corresponds to the initial length (l₀) of the specimen.

The instantaneous recovery Rᵢ is determined in the following manner:
the specimen is pulled by 30% (εₘₐₓ), i.e. about 0.3 times its initial length (l₀)
the constraint is released by applying a return speed equal to the tensile speed, i.e. 50 mm/min, and the residual elongation of the specimen is measured as a percentage, after returning to zero constraint (ε₁).

The percentage instantaneous recovery (R₁) is given by the following formula:

Rᵢ=(εₘₐₓ−εᵢ)/εₘₐₓ)×100

To determine the delayed recovery, the percentage residual elongation of the specimen (ε₂ₕ) is measured, 2 hours after returning to zero constraint.

The percentage delayed recovery (R₂ₕ) is given by the following formula:

R₂ₕ=(εₘₐₓ−ε₂ₕ)/εₘₐₓ)×100

Purely as a guide, a polymer according to one embodiment of the invention has an instantaneous recovery Rᵢ of 10% and a delayed recovery R₂ₕ of 30%.

Advantageously, the block polymer used in the composition according to the invention has a polydispersity index I of greater than 2, for example ranging from 2 to 9, preferably greater than or equal to 2.5, for example ranging from 2.5 to 8 and better still greater than or equal to 2.8, and especially ranging from 2.8 to 6.

The polydispersity index I of the block polymer is equal to the ratio of the weight-average mass Mw to the number-average mass Mn.

The weight-average molar mass (Mw) and number-average molar mass (Mn) are determined by gel permeation liquid chromatography (THF solvent, calibration curve established with linear polystyrene standards, refractometric detector).

The weight-average mass (Mw) of the block polymer is preferably less than or equal to 300 000; it ranges, for example, from 35 000 to 200 000 and better still from 45 000 to 150 000.

The number-average mass (Mn) of the block polymer is preferably less than or equal to 70 000; it ranges, for example, from 10 000 to 60 000 and better still from 12 000 to 50 000.

Each block of the block polymer of the composition according to the invention is derived from one type of monomer or from several different types of monomer.

This means that each block may consist of a homopolymer or a copolymer; this copolymer constituting the block may in turn be random or alternating.

Advantageously, the intermediate block comprising at least one constituent monomer of the first block and at least one constituent monomer of the second block of the polymer is a random polymer.

Preferably, the intermediate block is derived essentially from constituent monomers of the first block and of the second block.

The term “essentially” means at least 85%, preferably at least 90%, better still 95% and even better still 100%.

Advantageously, the intermediate block has a glass transition temperature Tg of between the glass transition temperatures of the first and second blocks.

The glass transition temperatures indicated for the first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$1 / Tg = {\sum }_{i} ( {ϖ}_{i} / {Tg}_{i} ) ,$$

ω̅ᵢ being the mass fraction of the monomer i in the block under consideration and Tgᵢ being the glass transition temperature of the homopolymer of the monomer i.

Unless otherwise indicated, the Tg values indicated for the first and second blocks in the present patent application are theoretical Tg values.

The difference between the glass transition temperatures of the first and second blocks is generally greater than 10° C., preferably greater than 20° C. and better still greater than 30° C.

In particular, the first block may be chosen from:
a) a block with a Tg of greater than or equal to 40° C.,
b) a block with a Tg of less than or equal to 20° C.,
c) a block with a Tg of between 20 and 40° C.,
   and the second block can be chosen from a category a), b) or c) different from the first block.

In the present invention, the expression: “between . . . and . . . ” is intended to denote a range of values for which the limits mentioned are excluded, and “from . . . to .” and “ranging from . . . to .” are intended to denote a range of values for which the limits are included.

a) Block with a Tg of Greater Than or Equal to 40° C.

The block with a Tg of greater than or equal to 40° C. has, for example, a Tg ranging from 40 to 150° C., preferably greater than or equal to 50° C., for example ranging from 50° C. to 120° C. and better still greater than or equal to 60° C., for example ranging from 60° C. to 120° C.

The block with a Tg of greater than or equal to 40° C. may be a homopolymer or a copolymer.

In the case where this block is a homopolymer, it is derived from monomers which are such that the homopolymers prepared from these monomers have glass transition temperatures of greater than or equal to 40° C. This first block may be a homopolymer consisting of only one type of monomer (for which the Tg of the corresponding homopolymer is greater than or equal to 40° C.).

In the case where the first block is a copolymer, it may be totally or partially derived from one or more monomers, the nature and concentration of which are chosen such that the Tg of the resulting copolymer is greater than or equal to 40° C. The copolymer may comprise, for example:
monomers which are such that the homopolymers prepared from these monomers have Tg values of greater than or equal to 40° C., for example a Tg ranging from 40 to 150° C., preferably greater than or equal to 50° C., for example ranging from 50° C. to 120° C. and better still greater than or equal to 60° C., for example ranging from 60° C. to 120° C., and
monomers which are such that the homopolymers prepared from these monomers have Tg values of less than 40° C., chosen from monomers with a Tg of between 20 and 40° C. and/or monomers with a Tg of less than or equal to 20° C., for example a Tg ranging from −100 to 20° C., preferably less than 15° C., especially ranging from −80° C. to 15° C. and better still less than 10° C., for example ranging from −50° C. to 0° C., as described later.

The monomers whose homopolymers have a glass transition temperature of greater than or equal to 40° C. are chosen, preferably, from the following monomers, also known as the main monomers:
methacrylates of formula CH₂═C(CH₃)—COOR₁ in which R₁ represents a linear or branched unsubstituted alkyl group containing from 1 to 4 carbon atoms, such as a methyl, ethyl, propyl or isobutyl group or R₁ represents a C₄ to C₁₂ cycloalkyl group,
acrylates of formula CH₂═CH—COOR₂ in which R₂ represents a C₄ to C₁₂ cycloalkyl group such as isobornyl acrylate or a tert-butyl group,
(meth)acrylamides of formula:

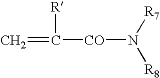

in which R₇ and R₈, which may be identical or different, each represent a hydrogen atom or a linear or branched C₁ to C₁₂ alkyl group such as an n-butyl, t-butyl, isopropyl, isohexyl, isooctyl or isononyl group; or R₇ represents H and R₈ represents a 1,1-dimethyl-3-oxobutyl group,
and R′ denotes H or methyl. Examples of monomers that may be mentioned include N-butylacrylamide, N-t-butyl-acrylamide, N-isopropylacrylamide, N,N-dimethyl-acrylamide and N,N-dibutylacrylamide,
and mixtures thereof.

Main monomers that are particularly preferred are methyl methacrylate, isobutyl(meth)acrylate and isobornyl(meth)acrylate, and mixtures thereof.

b) Block with a Tg of Less Than or Equal to 20° C.

The block with a Tg of less than or equal to 20° C. has, for example, a Tg ranging from −100 to 20° C., preferably less than or equal to 15° C., especially ranging from −80 to 15° C. and better still less than or equal to 10° C., for example ranging from −50 to 0° C.

The block with a Tg of less than or equal to 20° C. may be a homopolymer or a copolymer.

In the case where this block is a homopolymer, it is derived from monomers which are such that the homopolymers prepared from these monomers have glass transition temperatures of less than or equal to 20° C. This second block may be a homopolymer consisting of only one type of monomer (for which the Tg of the corresponding homopolymer is less than or equal to 20° C.).

In the case where the block with a Tg of less than or equal to 20° C. is a copolymer, it may be totally or partially derived from one or more monomers, the nature and concentration of which are chosen such that the Tg of the resulting copolymer is less than or equal to 20° C.

It may comprise, for example
one or more monomers whose corresponding homopolymer has a Tg of less than or equal to 20° C., for example a Tg ranging from −100 to 20° C., preferably less than 15° C., especially ranging from −80 to 15° C. and better still less than 10° C., for example ranging from −50° C. to 0° C., and
one or more monomers whose corresponding homopolymer has a Tg of greater than 20° C., such as monomers with a Tg of greater than or equal to 40° C., for example a Tg ranging from 40 to 150° C., preferably greater than or equal to 50° C., for example ranging from 50° C. to 120° C. and better still greater than or equal to 60° C., for example ranging from 60° C. to 120° C. and/or monomers with a Tg of between 20 and 40° C., as described above.

Preferably, the block with a Tg of less than or equal to 20° C. is a homopolymer.

The monomers whose homopolymer has a Tg of less than or equal to 20° C. are preferably chosen from the following monomers, or main monomers:
acrylates of formula CH₂═CHCOOR₃, R₃ representing a linear or branched C₁ to C₁₂ unsubstituted alkyl group, with the exception of the tert-butyl group, in which one or more hetero atoms chosen from O, N and S is (are) optionally intercalated,
methacrylates of formula CH₂═C(CH₃)—COOR₄, R₄ representing a linear or branched C₆ to C₁₂ unsubstituted alkyl group, in which one or more hetero atoms chosen from O, N and S is (are) optionally intercalated;
vinyl esters of formula R₅—CO—O—CH═CH₂ in which R₅ represents a linear or branched C₄ to C₁₂ alkyl group,
C₄ to C₁₂ alkyl vinyl ethers,
N—(C₄ to C₁₂)alkyl acrylamides, such as N-octylacrylamide,
and mixtures thereof.

The main monomers that are particularly preferred for the block with a Tg of less than or equal to 20° C. are alkyl acrylates whose alkyl chain contains from 1 to 10 carbon atoms, with the exception of the tert-butyl group, such as methyl acrylate, isobutyl acrylate and 2-ethylhexyl acrylate, and mixtures thereof.

c) Block with a Tg of Between 20 and 40° C.

The block with a Tg of between 20 and 40° C. may be a homopolymer or a copolymer.

In the case where this block is a homopolymer, it is derived from monomers (or main monomers) which are such that the homopolymers prepared from these monomers have glass transition temperatures of between 20 and 40° C. This first block may be a homopolymer, consisting of only one type of monomer (for which the Tg of the corresponding homopolymer ranges from 20° C. to 40° C.).

The monomers whose homopolymer has a glass transition temperature of between 20 and 40° C. are preferably chosen from n-butyl methacrylate, cyclodecyl acrylate, neopentyl acrylate and isodecylacrylamide, and mixtures thereof.

In the case where the block with a Tg of between 20 and 40° C. is a copolymer, it is totally or partially derived from one or more monomers (or main monomers) whose nature and concentration are chosen such that the Tg of the resulting copolymer is between 20 and 40° C.

Advantageously, the block with a Tg of between 20 and 40° C. is a copolymer totally or partially derived from:
main monomers whose corresponding homopolymer has a Tg of greater than or equal to 40° C., for example a Tg ranging from 40° C. to 150° C., preferably greater than or equal to 50° C., for example ranging from 50 to 120° C. and better still greater than or equal to 60° C., for example ranging from 60° C. to 120° C., as described above, and/or
main monomers whose corresponding homopolymer has a Tg of less than or equal to 20° C., for example a Tg ranging from −100 to 20° C., preferably less than or equal to 15° C., especially ranging from −80° C. to 15° C. and better still less than or equal to 10° C., for example ranging from −50° C. to 0° C., as described above, the said monomers being chosen such that the Tg of the copolymer forming the first block is between 20 and 40° C.

Such main monomers are chosen, for example, from methyl methacrylate, isobornyl acrylate and methacrylate, butyl acrylate and 2-ethylhexyl acrylate, and mixtures thereof.

Preferably, the proportion of the second block with a Tg of less than or equal to 20° C. ranges from 10% to 85% by weight, better still from 20% to 7-0% and even better still from 20% to 50% by weight of the polymer.

However, each of the blocks may contain in small proportion at least one constituent monomer of the other block.

Thus, the first block may contain at least one constituent monomer of the second block, and vice versa.

Each of the first and/or second blocks may comprise, in addition to the monomers indicated above, one or more other monomers known as additional monomers, which are different from the main monomers mentioned above.

The nature and amount of this or these additional monomer(s) are chosen such that the block in which they are present has the desired glass transition temperature.

This additional monomer is chosen, for example, from:

hydrophilic monomers such as:
ethylenically unsaturated monomers comprising at least one carboxylic or sulphonic acid function, for instance:
   acrylic acid, methacrylic acid, crotonic acid, maleic anhydride, itaconic acid, fumaric acid, maleic acid, acrylamidopropanesulphonic acid, vinylbenzoic acid, vinylphosphoric acid, and salts thereof,
ethylenically unsaturated monomers comprising at least one tertiary amine function, for instance 2-vinylpyridine, 4-vinylpyridine, dimethyl-aminoethyl methacrylate, diethylaminoethyl methacrylate and dimethylaminopropylmethacrylamide, and salts thereof,
methacrylates of formula CH₂═C(CH₃)—COOR₆ in which R₆ represents a linear or branched alkyl group containing from 1 to 4 carbon atoms, such as a methyl, ethyl, propyl or isobutyl group, the said alkyl group being substituted with one or more substituents chosen from hydroxyl groups (for instance 2-hydroxypropyl methacrylate and 2-hydroxyethyl methacrylate) and halogen atoms (Cl, Br, I or F), such as trifluoroethyl methacrylate,
methacrylates of formula CH₂═C(CH₃)—COOR₉, R₉ representing a linear or branched C₆ to C₁₂ alkyl group in which one or more hetero atoms chosen from O, N and S is (are) optionally intercalated, the said alkyl group being substituted with one or more substituents chosen from hydroxyl groups and halogen atoms (Cl, Br, I or F);
acrylates of formula CH₂═CHCOOR₁₀, R₁₀ representing a linear or branched C₁ to C₁₂ alkyl group substituted with one or more substituents chosen from hydroxyl groups and halogen atoms (Cl, Br, I or F), such as 2-hydroxypropyl acrylate and 2-hydroxyethyl acrylate, or R₁₀ represents a C₁ to C₁₂ alkyl-O—POE (polyoxyethylene) with repetition of the oxyethylene unit 5 to 30 times, for example methoxy-POE, or R₈ represents a polyoxyethylene group containing from 5 to 30 ethylene oxide units

b) ethylenically unsaturated monomers comprising one or more silicon atoms, such as methacryloxypropyltrimethoxysilane and methacryloxypropyltris(trimethylsiloxy)silane,
and mixtures thereof.

Additional monomers that are particularly preferred are acrylic acid, methacrylic acid and trifluoroethyl methacrylate, and mixtures thereof.

According to one preferred embodiment, the block polymer is a non-silicone polymer, i.e. a polymer free of silicon atoms.

This or these additional monomer(s) generally represent(s) an amount of less than or equal to 30% by weight, for example from 1% to 30% by weight, preferably from 5% to 20% by weight and more preferably from 7% to 15% by weight, relative to the total weight of the first and/or second blocks.

Preferably, each of the first and second blocks comprises at least one monomer chosen from (meth)acrylic acid esters, and optionally at least one monomer chosen from (meth)acrylic acid, and mixtures thereof.

Advantageously, each of the first and second blocks is derived entirely from at least one monomer chosen from acrylic acid, (meth)acrylic acid esters and optionally from at least one monomer chosen from (meth)acrylic acid, and mixtures thereof.

The block polymer may be obtained by free-radical solution polymerization according to the following preparation process:
a portion of the polymerization solvent is introduced into a suitable reactor and heated until the adequate temperature for the polymerization is reached (typically between 60 and 120° C.),
once this temperature is reached, the constituent monomers of the first block are introduced in the presence of some of the polymerization initiator,
after a time T corresponding to a maximum degree of conversion of 90%, the constituent monomers of the second block and the rest of the initiator are introduced,
the mixture is left to react for a time T′ (ranging from 3 to 6 hours), after which the mixture is cooled to room temperature,
the polymer dissolved in the polymerization solvent is obtained.

The term polymerization solvent means a solvent or a mixture of solvents. The polymerization solvent may be chosen in particular from ethyl acetate, butyl acetate, alcohols such as isopropanol, ethanol, aliphatic alkanes such as isododecane and mixtures thereof. Preferably, the polymerization solvent is a mixture of butyl acetate and isopropanol or isododecane.

### FIRST EMBODIMENT

According to a first embodiment, the block polymer comprises a first block with a Tg of greater than or equal to 40° C., as described above in a) and a second block with a Tg of less than or equal to 20° C., as described above in b).

Preferably, the first block with a Tg of greater than or equal to 40° C. is a copolymer derived from monomers which are such that the homopolymer prepared from these monomers has a glass transition temperature of greater than or equal to 40° C., such as the monomers described above.

Advantageously, the second block with a Tg of less than or equal to 20° C. is a homopolymer derived from monomers which are such that the homopolymer prepared from these monomers has a glass transition temperature of less than or equal to 20° C., such as the monomers described above.

Preferably, the proportion of the block with a Tg of greater than or equal to 40° C. ranges from 20% to 90%, better still from 30% to 80% and even better still from 50% to 70% by weight of the polymer. Preferably, the proportion of the block with a Tg of less than or equal to 20° C. ranges from 5% to 75%, preferably from 15% to 50% and better still from 25% to 45% by weight of the polymer.

Advantageously, the block polymer may comprise:
a first block with a Tg of greater than or equal to 40° C., for example ranging from 85 to 115° C., which is an isobornyl acrylate/isobutyl methacrylate copolymer,
a second block with a Tg of less than or equal to 20° C., for example ranging from −85 to −55° C., which is a 2-ethylhexyl acrylate homopolymer, and
an intermediate block, which is an isobornyl acrylate/isobutyl methacrylate/2-ethylhexyl acrylate random copolymer.

### SECOND EMBODIMENT

According to a second embodiment, the block polymer comprises a first block having a glass transition temperature (Tg) of between 20 and 40° C., in accordance with the blocks described in c) and a second block having a glass transition temperature of less than or equal to 20° C., as described above in b) or a glass transition temperature of greater than or equal to 40° C., as described in a) above.

Preferably, the proportion of the first block with a Tg of between 20 and 40° C. ranges from 10% to 85%, better still from 30% to 80% and even better still from 50% to 70% by weight of the polymer.

When the second block is a block with a Tg of greater than or equal to 40° C., it is preferably present in a proportion ranging from 10% to 65% by weight, better still from 20% to 70% and even better still from 30% to 70% by weight of the polymer.

When the second block is a block with a Tg of less than or equal to 20° C., it is preferably present in a proportion ranging from 10% to 85% by weight, better still from 20% to 70% and even better still from 20% to 50% by weight of the polymer.

Preferably, the first block with a Tg of between 20 and 40° C. is a copolymer derived from monomers which are such that the corresponding homopolymer has a Tg of greater than or equal to 40° C., and from monomers which are such that the corresponding homopolymer has a Tg of less than or equal to 20° C.

Advantageously, the second block with a Tg of less than or equal to 20° C. or with a Tg of greater than or equal to 40° C. is a homopolymer.

According to a first variant, the block polymer comprises:
a first block with a Tg of between 20 and 40° C., for example with a Tg of 21 to 39° C., which is a copolymer comprising isobornyl acrylate/isobutyl methacrylate/2-ethylhexyl acrylate,
a second block with a Tg of less than or equal to 20° C., for example ranging from −65 to −35° C., which is a methyl methacrylate homopolymer, and
an intermediate block which is an isobornyl acrylate/isobutyl methacrylate/2-ethylhexyl acrylate random copolymer.

According to a second variant, the polymer according to the invention may comprise:
a first block with a Tg of greater than or equal to 40° C., for example ranging from 85 to 115° C., which is an isobornyl methacrylate/isobutyl methacrylate copolymer,
a second block with a Tg of less than or equal to 20° C., for example ranging from −35 to −5° C., which is an isobutyl acrylate homopolymer, and
an intermediate block which is an isobornyl methacrylate/isobutyl methacrylate/isobutyl acrylate random copolymer.

According to a third variant, the polymer according to the invention may comprise:
a first block with a Tg of greater than or equal to 40° C., for example ranging from 60 to 90° C., which is an isobornyl acrylate/isobutyl methacrylate copolymer,
a second block with a Tg of less than or equal to 20° C., for example ranging from −35 to −5° C., which is an isobutyl acrylate homopolymer, and
an intermediate block which is an isobornyl acrylate/isobutyl methacrylate/isobutyl acrylate random copolymer.

According to one embodiment, the composition according to the invention may comprise a mixture of two ethylenic block polymers chosen from the polymers described above.

The block polymer(s) may be present in the composition according to the invention in a dry matter (or active material) content ranging from 5 to 55%, preferably ranging from 6 to 45% and better still from 8 to 40% by weight relative to the total weight of the composition.

### 2) Semicrystalline Polymer

The term “semi-crystalline polymer” means within the context of the invention polymers comprising a crystallizable portion, a crystallizable pendent chain or a crystallizable block in the skeleton, and an amorphous portion in the skeleton and having a temperature of first-order reversible phase change, in particular of melting (solid-liquid transition). When the crystallizable portion is in the form of a crystallizable block of the polymer skeleton, the amorphous portion of the polymer is in the form of an amorphous block; in this case, the semi-crystalline polymer is a block copolymer, for example of the diblock, triblock or multiblock type, comprising at least one crystallizable block and at least one amorphous block. The term “block” generally means at least 5 identical repeating units. The crystallizable block(s) are then of different chemical nature from the amorphous block(s).

The semi-crystalline polymer according to the invention has a melting point of greater than or equal to 30° C. (especially ranging from 30° C. to 60° C.) and preferably ranging from 30° C. to 60° C. This melting point is a temperature of first-order change of state.

This melting point may be measured by any known method, and in particular via differential scanning calorimetry (DSC).

Advantageously, the semi-crystalline polymer(s) to which the invention applies have a number-average molecular mass of greater than or equal to 1000.

Advantageously, the semi-crystalline polymer(s) of the composition of the invention have a number-average molecular mass M̅n ranging from 200-0 to 800 000, preferably from 3000 to 500 000, better still from 4000 to 150 000, especially less than 100 000 and better still from 4000 to 99 000. They preferably have a number-average molecular mass of greater than 5600, for example ranging from 5700 to 99 000.

For the purposes of the invention, the expression “crystallizable chain or block” means a chain or block which, if it were obtained alone, would change from the amorphous state to the crystalline state reversibly, depending on whether one is above or below the melting point. For the purposes of the invention, a “chain” is a group of atoms, which are pendent or lateral relative to the polymer skeleton. A “block” is a group of atoms belonging to the skeleton, this group constituting one of the repeating units of the polymer. Advantageously, the “pendent crystallizable chain” may be a chain containing at least 6 carbon atoms.

Preferably, the crystallizable blocks) or chains) of the semi-crystalline polymers represent at least 30% of the total weight of each polymer and better still at least 40%. The semi-crystalline polymers of the invention containing crystallizable blocks are block or multiblock polymers. They may be obtained by polymerizing a monomer containing reactive (or ethylenic) double bonds or by polycondensation. When the polymers of the invention are polymers containing crystallizable side chains, these side chains are advantageously in random or statistical form.

Preferably, the semi-crystalline polymers of the invention are of synthetic origin. Moreover, they do not comprise a polysaccharide skeleton. In general, the crystallizable units (chains or blocks) of the semi-crystalline polymers according to the invention originate from monomer(s) containing crystallizable block(s) or chain(s), used for the manufacture of the semi-crystalline polymers.

According to the invention, the semicrystalline polymer may be chosen from the block copolymers comprising at least one crystallizable block and at least one amorphous block, the homopolymers and the copolymers bearing at least one crystallizable side chain per repeating unit, mixtures thereof.

The semi-crystalline polymers that may be used in the invention are, in particular:
block copolymers of polyolefins with controlled crystallization, especially those whose monomers are described in EP-A-0 951 897,
polycondensates, especially of aliphatic or aromatic polyester type or of aliphatic/aromatic copolyester type,
homopolymers or copolymers bearing at least one crystallizable side chain and homopolymers or copolymers bearing at least one crystallizable block in the skeleton, for instance those described in document U.S. Pat. No. 5,156,911,
homopolymers or copolymers bearing at least one crystallizable side chain, in particular containing fluoro group(s), as described in document WO-A-01/19333,
   and mixtures thereof. In the last two cases, the crystallizable side chain(s) or block(s) are hydrophobic.

### A) Semi-Crystalline Polymers Containing Crystallizable Side Chains

Mention may be made in particular of those defined in documents U.S. Pat. No. 5,156,911 and WO-A-01/19333. They are homopolymers or copolymers comprising from 50% to 100% by weight of units resulting from the polymerization of one or more monomers bearing a crystallizable hydrophobic side chain. These homopolymers or copolymers are of any nature, provided that they meet the conditions mentioned previously.

They can result:
from the polymerization, especially the free-radical polymerization, of one or more monomers containing reactive or ethylenic double bond(s) with respect to a polymerization, namely a vinyl, (meth)acrylic or allylic group,
from the polycondensation of one or more monomers bearing co-reactive groups carboxylic acid, sulphonic acid, alcohol, amine or isocyanate), such as, for example, polyesters, polyurethanes, polyethers, polyureas or polyamides.

In general, these polymers are chosen especially from homopolymers and copolymers resulting from the polymerization of at least one monomer containing crystallizable chain(s) that may be represented by formula X:

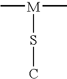

with M representing an atom of the polymer skeleton, S representing a spacer and C representing a crystallizable group.

The crystallizable chains “—S—C” may be aliphatic or aromatic, and optionally fluorinated or perfluorinated. “S” especially represents a group (CH₂)ₙ or (CH₂CH₂O)ₙ or (CH₂O), which may be linear or branched or cyclic, with n being an integer ranging from 0 to 22. Preferably, “S” is a linear group. Preferably, “S” and “C” are different.

When the crystallizable chains “—S—C” are hydrocarbon-based aliphatic chains, they comprise hydrocarbon-based alkyl chains containing at least 11 carbon atoms and not more than 40 carbon atoms and better still not more than 24 carbon atoms. They are especially aliphatic chains or alkyl chains containing at least 12 carbon atoms, and they are preferably C₁₄-C₂₄ alkyl chains. When they are fluoroalkyl or perfluoroalkyl chains, they contain at least 6 fluorinated carbon atoms and especially at least 11 carbon atoms, at least 6 of which carbon atoms are fluorinated.

As examples of semi-crystalline polymers or copolymers containing crystallizable chains), mention may be made of those resulting from the polymerization of one or more of the following monomers: (meth)acrylates of saturated alkyl with the alkyl group being C₁₄-C₂₄, perfluoroalkyl(meth)acrylates with a C₁₁-C₁₅ perfluoroalkyl group, N-alkyl(meth)acrylamides with the alkyl group being C₁₄ to C₂₄ with or without a fluorine atom, vinyl esters containing alkyl or perfluoro(alkyl) chains with the alkyl group being C₁₄ to C₂₄ (with at least 6 fluorine atoms per perfluoroalkyl chain), vinyl ethers containing alkyl or perfluoro(alkyl) chains with the alkyl group being C₁₄ to C₂₄ and at least 6 fluorine atoms per perfluoroalkyl chain, C₁₄ to C₂₄ alpha-olefins such as, for example, octadecene, para-alkylstyrenes with an alkyl group containing from 12 to 24 carbon atoms, and mixtures thereof.

When the polymers result from a poly-condensation, the hydrocarbon-based and/or fluorinated crystallizable chains as defined above are borne by a monomer that may be a diacid, a diol, a diamine or a diisocyanate.

When the polymers that are the subject of the invention are copolymers, they additionally contain from 0 to 50% of groups Y or Z resulting from the copolymerization:

α) of Y which is a polar or non-polar monomer or a mixture of the two:
When Y is a polar monomer, it is either a monomer bearing polyoxyalkylenated groups (especially oxyethylenated and/or oxypropylenated groups), a hydroxyalkyl(meth)acrylate, for instance hydroxyethyl acrylate, (meth)acrylamide, an N-alkyl(meth)acrylamide, an N,N-dialkyl(meth)acrylamide such as, for example, N,N-diisopropylacrylamide or N-vinylpyrrolidone (NVP), N-vinylcaprolactam, a monomer bearing at least one carboxylic acid group, for instance (meth)acrylic acid, crotonic acid, itaconic acid, maleic acid or fumaric acid, or bearing a carboxylic acid anhydride group, for instance maleic anhydride, and mixtures thereof.
When Y is a non-polar monomer, it may be an ester of the linear, branched or cyclic alkyl (meth)acrylate type, a vinyl ester, an alkyl vinyl ether, an alpha-olefin, styrene or styrene substituted with a C₁ to C₁₀ alkyl group, for instance α-methyl-styrene, or a macromonomer of the polyorganosiloxane type containing vinyl unsaturation.

For the purposes of the invention, the term “alkyl” means a saturated group especially of C₈ to C₂₄, except where otherwise mentioned, and better still of C₁₄ to C₂₄.

∃) of Z which is a polar monomer or a mixture of polar monomers. In this case, Z has the same definition as the “polar Y” defined above.

Preferably, the semi-crystalline polymers containing a crystallizable side chain are alkyl (meth)acrylate or alkyl(meth)acrylamide homopolymers with an alkyl group as defined above, and especially of C₁₄-C₂₄, copolymers of these monomers with a hydrophilic monomer preferably of different nature from (meth)acrylic acid, for instance N-vinylpyrrolidone or hydroxyethyl(meth)acrylate, and mixtures thereof.

### B) Polymers Bearing in the Skeleton at Least One Crystallizable Block

These polymers are especially block copolymers consisting of at least 2 blocks of different chemical nature, one of which is crystallizable.
The block polymers defined in U.S. Pat. No. 5,156,911 may be used;
Block copolymers of olefin or of cycloolefin containing a crystallizable chain, for instance those derived from the block polymerization of:
cyclobutene, cyclohexene, cyclooctene, norbornene (i.e. bicyclo(2,2,1)-2-heptene), 5-methyl-norbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinyl-norbornene, 1,4,5,8-dimethano-1,2,3,4,4a,5,8a-octa-hydronaphthalene, dicyclopentadiene, or mixtures thereof,
with ethylene, propylene, 1-butene, 3-methyl-1-butene, 1-hexene, 4-methyl-1-pentene, 1-octene, 1-decene or 1-eicosene, or mixtures thereof,
and in particular copoly(ethylene/norbornene) blocks and (ethylene/propylene/ethylidene-norbornene) block terpolymers. Those resulting from the block copolymerization of at least 2 C₂-C₁₆, better still C₂-C₁₂ and even better still C₄-C₁₂ α-olefins such as those mentioned above and in particular block bipolymers of ethylene and of 1-octene may also be used.
The copolymers may be copolymers containing at least one crystallizable block, the copolymer residue being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:
Block that is crystallizable by nature: a) polyester, for instance poly(alkylene terephthalate), b) polyolefin, for instance polyethylenes or polypropylenes.
Amorphous and lipophilic block, for instance amorphous polyolefins or copoly(olefin)s such as poly(isobutylene), hydrogenated polybutadiene or hydrogenated poly(isoprene).

As examples of such copolymers containing a crystallizable block and a separate amorphous block, mention may be made of:

α) poly(ε-caprolactone)-b-poly(butadiene) block copolymers, preferably used hydrogenated, such as those described in the article “Melting behavior of poly(ε-caprolactone)-block-polybutadiene copolymers” from S. Nojima, Macromolecules, 32, 3727-3734 (1999),

∃) the hydrogenated block or multiblock poly(butylene terephthalate)-b-polyisoprene) block copolymers cited in the article “Study of morphological and mechanical properties of PP/PBT” by B. Boutevin et al., Polymer Bulletin, 34, 117-123 (1995),
( ) the poly(ethylene)-b-copolylethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),
δ) the poly(ethylene)-b-polylethylethylene) block copolymers cited in the general article “Crystallization in block copolymers” by I. W. Hamley, Advances in Polymer Science, Vol. 148, 113-137 (1999).

The semi-crystalline polymers in the composition of the invention may or may not be partially crosslinked, provided that the degree of crosslinking does not interfere with their dissolution or dispersion in the liquid fatty phase by heating above their melting point. It may then be a chemical crosslinking, by reaction with a multifunctional monomer during the polymerization. It may also be a physical crosslinking which may, in this case, be due either to the establishment of bonds of hydrogen or dipolar type between groups borne by the polymer, such as, for example, the dipolar interactions between carboxylate ionomers, these interactions being of small amount and borne by the polymer skeleton; or to a phase separation between the crystallizable blocks and the amorphous blocks borne by the polymer.

Preferably, the semi-crystalline polymers in the composition according to the invention are non-crosslinked.

According to one particular embodiment of the invention, the polymer is chosen from copolymers resulting from the polymerization of at least one monomer containing a crystallizable chain Chosen from saturated C₁₄ to C₂₄ alkyl(meth)acrylates, C₁₁ to C₁₅ perfluoroalkyl(meth)acrylates, C₁₄ to C₂₄ N-alkyl(meth)-acrylamides with or without a fluorine atom, vinyl esters containing C₁₄ to C₂₄ alkyl or perfluoroalkyl chains, vinyl ethers containing C₁₄ to C₂₄ alkyl or perfluoroalkyl chains, C₁₄ to C₂₄ alpha-olefins, para-alkylstyrenes with an alkyl group containing from 12 to 24 carbon atoms, with at least one optionally fluorinated C₁₀ to C₁₀ monocarboxylic acid ester or amide, which may be represented by the following formula:

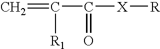

in which R₁ is H or CH₃, R represents an optionally fluorinated C₁-C₁₀ alkyl group and X represents O, NH or NR₂ in which R₂ represents an optionally fluorinated C₁-C₁₀ alkyl group.

According to one more particular embodiment of the invention, the polymer is derived from a monomer containing a crystallizable chain, chosen from saturated C₁₄ to C₂₂ alkyl(meth)acrylates.

As specific examples of the structuring semi-crystalline polymers that may be used in the composition according to the invention, mention may be made of the products Intelimer® from the company Landec, described in the brochure “Intelimer® polymers”, Landec IP22 (Rev. 4-97). These polymers are in solid form at room temperature (25° C.). They bear crystallizable side chains and have the formula X above.

The semi-crystalline polymers may especially be: those described in Examples 3, 4, 5, 7, 9 and 13 of U.S. Pat. No. 5,156,911 containing a —COOH group, resulting from the copolymerization of acrylic acid and of C₅ to C₁₆ alkyl(meth)acrylate and more particularly from the copolymerization:
of acrylic acid, of hexadecyl acrylate and of isodecyl acrylate in a 1/1.6/3 weight ratio,
of acrylic acid and of pentadecyl acrylate in a 1/19 weight ratio,
of acrylic acid, of hexadecyl acrylate and of ethyl acrylate in a 2.5/76.5/20 weight ratio,
of acrylic acid, of hexadecyl acrylate and of methyl acrylate in a 5/85/10 weight ratio,
of acrylic acid and of octadecyl methacrylate in a 2.5/97.5 weight ratio,
of hexadecyl acrylate, of polyethylene glycol methacrylate monomethyl ether containing 8 ethylene glycol units, and of acrylic acid in an 8.5/1/0.5 weight ratio.

It is also possible to use the structure “O” polymer from National Starch, as described in document U.S. Pat. No. 5,736,125, with a melting point of 44° C., and also semi-crystalline polymers with crystallizable pendent chains comprising fluoro groups, as described in Examples 1, 4, 6, 7 and 8 of document WO-A-01/19333.

It is also possible to use semi-crystalline polymers obtained by copolymerization of stearyl acrylate and of acrylic acid or of NVP, as described in document U.S. Pat. No. 5,519,063 or EP-A-550 745, with a melting point of 40° C. and 38° C., respectively.

It is also possible to use semi-crystalline polymers obtained by copolymerization of behenyl acrylate and of acrylic acid or of NVP, as described in documents U.S. Pat. No. 5,519,063 and EP-A-550 745, with a melting point of 60° C. and 58° C., respectively.

Preferably, the semicrystalline polymers do not contain a carboxylic group.

The semicrystalline polymer may be present in a dry matter content ranging from 0.1% to 15% by weight, preferably from 0.5% to 40% by weight, and better still from 1% to 30% by weight-relative to the total weight of the composition.

### 3) Cosmetically Acceptable Organic Liquid Medium

The term “organic liquid medium” means a medium containing at least one-organic compound that is liquid at room temperature (25° C.) and atmospheric pressure (10⁵ Pa) such as the organic oils and solvents commonly used in cosmetic compositions.

According to a particularly preferred embodiment, the organic liquid medium of the composition contains at least one organic liquid which is the or one of the organic solvent(s) for polymerizing the block polymer as described above. Advantageously, the said organic polymerization solvent is the major organic liquid by weight in the organic liquid medium of the cosmetic composition.

The organic liquid medium of the composition may represent from 10 to 95%, preferably from 20 to 90%, and better still from 30 to 80% by weight relative to the total weight of the composition.

The organic oils or solvents can form especially a fatty phase, and in particular a continuous fatty phase. The composition may be an anhydrous composition.

The cosmetically acceptable organic liquid medium of the composition advantageously comprises at least one volatile organic solvent or oil defined below.

For the purposes of the invention, the expression “volatile organic solvent or oil” means any non-aqueous medium that can evaporate on contact with the keratin fibre in less than one hour at room temperature and atmospheric pressure. The volatile organic solvent(s) and the volatile oils of the invention are organic solvents and volatile cosmetic oils, that are liquid at room temperature, having a non-zero vapour pressure at room temperature and atmospheric pressure, ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg). The expression “non-volatile oil” means an oil that remains on the keratin fibre at room temperature and atmospheric pressure for at least several hours and which in particular has a vapour pressure of less than 10⁻³ mmHg (0.13 Pa).

These oils may be hydrocarbon-based oils, silicone oils, or mixtures thereof.

The expression “hydrocarbon-based oil” means an oil mainly containing hydrogen and carbon atoms and optionally oxygen, nitrogen, sulphur or phosphorus atoms. The volatile hydrocarbon-based oils may be chosen from hydrocarbon-based oils containing from 6 to 16 carbon atoms, and especially C₈-C₁₆ branched alkanes, for instance C₈-C₁₆ isoalkanes of petroleum origin (also known as isoparaffins), for instance isododecane (also known as 2,2,4,4,6-pentamethylheptane), isodecane and isohexadecane, and, for example, the oils sold under the trade names Isopars or Permethyls, C₈-C₁₆ branched esters, isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from hydrocarbon-based volatile oils containing from 8 to 16 carbon atoms, and mixtures thereof.

Volatile oils which may also be used are volatile silicones such as, for example, linear or cyclic volatile silicone oils, especially those with a viscosity≦6 centistokes (6×10⁻⁶ m²/s) and especially containing from 2 to 10 silicon atoms, these silicones optionally comprising alkyl or alkoxy groups containing from 1 to 22 carbon atoms. As volatile silicone oils which may be used in the invention, mention may be made in particular of octamethylcyclotetrasiloxane, deca-methylcyclopentasiloxane, dodecamethylcyclohexa-siloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The volatile oil may be present in the composition according to the invention in a content ranging from 0.5% to 95% by weight and preferably from 1 to 65% by weight and better still from 5 to 40% by weight relative to the total weight of the composition.

The non-volatile silicone oils which may be used in the composition according to the invention may be non-volatile polydimethylsiloxanes (EDMSs), poly-dimethylsiloxanes comprising alkyl or alkoxy groups, that are pendent and/or at the end of a silicone chain, the groups each containing from 2 to 24 carbon atoms, phenylsilicones, for instance phenyltrimethicones, phenyldimethicones, phenyltrimethylsilvxydiphenyl-siloxanes, diphenyldimethicones, diphenylmethyl-diphenyltrisiloxanes and 2-phenylethyl trimethylsiloxy-silicates.

The fluoro oils which can be used in the composition of the invention are especially fluoro-silicone oils, polyfluoro ethers, fluorosilicones as described in the document EP-A-847752.

The non-volatile oils may be present in the composition according to the invention in a content ranging from 0 to 30% (especially from 0.1 to 30%) by weight, preferably from 0 to 20% by weight (especially 0.1 to 20%) and better still from 0 to 10% by weight (especially 0.1% to 10%), relative to the total weight of the composition.

In one embodiment of the invention, the organic liquid medium of the composition-comprises at least one volatile organic oil which is the solvent for polymerizing the block polymer and in which the block polymer is advantageously soluble. Preferably, this volatile organic oil is isododecane. Such a composition has the advantage of being easy to remove when used as makeup, with a conventional makeup-removing product for waterproof mascaras.

Advantageously, the composition according to the invention comprises an aqueous medium, constituting an aqueous phase, which can form the continuous phase of the composition.

The aqueous phase may consist mainly of water; it may also comprise a mixture of water and a water-miscible solvent (miscibility in water greater than 50% by weight at 25° C.) such as lower monoalcohols having from 1 to 5 carbon atoms such as ethanol, isopropanol, glycols having from 2 to 8 carbon atoms such as propylene glycol, ethylene glycol, 1,3-butylene glycol, dipropylene glycol, C₃-C₄ ketones, C₂-C₄ aldehydes and mixtures thereof.

The aqueous phase (water and optionally the water-miscible solvent) may be present in a content ranging from 1% to 95% by weight, preferably ranging from 3% to 80% by weight, and preferentially ranging from 5% to 60% by weight, relative to the total weight of the composition.

### Wax

The composition according to the invention may comprise a wax or a mixture of waxes.

The wax under consideration in the context of the present invention is generally a lipophilic compound that is solid at room temperature (25° C.), with a solid/liquid reversible change of state, having a melting point of greater than or equal to 30° C., which may be up to 120° C.

By bringing the wax to the liquid form (melting), it is possible to make it miscible with oils and to form a microscopically uniform mixture, but on bringing the mixture back to room temperature, recrystallization of the wax in the oils of the mixture is obtained.

In particular, the waxes that are suitable for the invention may have a melting point of greater than about 45° C. and in particular greater than −55° C.

The melting point of the wax may be measured using a differential scanning calorimeter (DSC), for example the calorimeter sold under the name DSC 30 by the company Metler.

The measuring protocol is as follows:

A sample of 15 mg of product placed in a crucible is subjected to a first temperature rise ranging from 0° C. to 120° C., at a heating rate of 10° C./minute, it is then cooled from 120° C. to 0°, at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding too the top of the peak of the curve representing the variation in the difference in absorbed power as a function of the temperature.

The waxes that may be used in the compositions according to the invention are chosen from waxes that are solid and rigid at room temperature, of animal, plant, mineral or synthetic origin and mixtures thereof.

The wax may also have a hardness ranging from 0.05 MPa to 30 M-pa, preferably ranging from 6 MPa to 15 MPa. The hardness is determined by measuring the compression force, measured at 20° C. using a texturometer sold under the name TA-TX2i by the company Rheo, equipped with a stainless-steel Cylindrical spindle 2 mm in diameter, travelling at a measuring speed of 0.1 mm/s, and penetrating into the wax to a penetration depth of 0.3 mm.

The measuring protocol is as follows:

The wax is melted at a temperature equal to the melting point of the wax +20° C. The molten wax is poured into a container 30 mm in diameter and 20 mm deep. The wax is recrystallized at room temperature (25° C.) for 24 hours and is then stored for at least 1 hour at 20° C., before performing the hardness measurement. The hardness value is the maximum compression force measured, divided by the area of the texturometer spindle in contact with the wax.

Hydrocarbon-based waxes, for instance beeswax, lanolin wax, Chinese insect waxes, rice wax, carnauba wax, candelilla wax, ouricurry wax, esparto grass wax, cork fibre wax, sugar cane wax, Japan wax and sumac wax; montan wax, microcrystalline waxes, paraffins and ozokerite; polyethylene waxes, the waxes obtained by Fischer-Tropsch synthesis and waxy copolymers, and also esters thereof, may especially be used.

Mention may also be made of waxes obtained by catalytic hydrogenation of animal or plant oils containing linear or branched C₈-C₃₂ fatty chains.

Among these, mention may be made especially of hydrogenated jojoba oil, isomerized jojoba oil such as the partially hydrogenated trans-isomerized jojoba oil manufactured or sold by the company Desert Whale under the commercial reference Iso-Jojoba-50®, hydrogenated sunflower oil, hydrogenated castor oil, hydrogenated coconut oil and hydrogenated lanolin oil, bis(1,1,1-trimethylolpropane) tetrastearate sold under the name “Hest 2T-4S” by the company Heterene and bis(1,1,1-trimethylolpropane) tetrabehenate sold under the name Hest 2T-4B by the company Heterene.

Mention may also be made of silicone waxes and fluoro waxes.

It is also possible to use the wax obtained by hydrogenation of olive oil esterified with stearyl alcohol, sold under the name “Phytowax Olive 18 L 57” or the waxes obtained by hydrogenation of castor oil esterified with cetyl alcohol, sold under the name “Phytowax Ricin 16L64 and 22L73” by the company Sophim. Such waxes are described in patent application FR-A-2 792 190.

The composition according to the invention may comprise a total wax content ranging from 1 to 50% by weight, in particular it may comprise from −5 to 30% by weight, and more particularly from 10 to 30% by weight relative to the total weight of the composition.

The wax(es) may be in the form of an aqueous microdispersion of wax. The expression “aqueous microdispersion of wax” means an aqueous dispersion of wax particles in which the size of the said wax particles is less than or equal to about 1 μm.

Wax microdispersions are stable dispersions of colloidal wax particles, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of a portion of water, followed by gradual addition of hot-water with stirring. The intermediate formation of an emulsion of the water-in-oil type is observed, followed by a phase inversion, with final production of a microemulsion of the oil-in-water type. On cooling, a stable microdispersion of solid wax colloidal particles is obtained.

The wax microdispersions may also be obtained by stirring the mixture of wax, surfactant and water using stirring means such as ultrasound, high-pressure homogenizers or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles consist essentially of a wax or a mixture of waxes. However, they may comprise a small proportion of oily and/or pasty fatty additives, a surfactant and/or a common liposoluble additive/active agent.

In some cases and depending on the wishes of consumers, it is desirable to prepare cosmetic compositions having the advantages described above and having a glossy appearance. Accordingly, another subject of the present invention is a wax-free composition for coating keratin fibres, comprising a cosmetically acceptable liquid organic medium, a firm-forming linear ethylenic block polymer and a semicrystalline polymer.

Indeed, the use of a semicrystalline polymer makes it possible to structure the composition without resorting to a wax and to keep the consistency of the said composition flexible.

The expression “wax-free” means a composition comprising less than 2% of wax, preferably less than 1% and better still less than 0.5% of wax.

Such a wax-free composition also has the advantage of allowing a deposit to be obtained that is particularly smooth, homogeneous and nongranular.

Another subject of the present invention is the use of a wax-free composition for coating keratin fibres comprising a cosmetically acceptable liquid organic medium, a film-forming linear ethylenic block polymer and a semicrystalline polymer to obtain a film, deposited on the said keratin materials, that is smooth and homogeneous and has a glossy appearance.

Such a wax-free composition may be especially used as topcoat, i.e. as a composition to be applied over a mascara base coat (basecoat) so as to improve the staying power of the said mascara.

The composition according to the invention may contain at least one-fatty compound that is pasty at room temperature. For the purposes of the invention, the expression “pasty fatty substance” weans fatty substances with a melting point ranging from 20 to 55° C., preferably 25 to 45° C., and/or a viscosity at 40° C., ranging from 0.1 to 40 Pa·s (1 to 400 poises), preferably 0.5 to 25 Pa·s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to carry out the measurement of the pasty compound tested.

These fatty substances are preferably hydrocarbon-based compounds, optionally of polymeric type; they can also be chosen from silicone compounds; they may also be in the form of a mixture of hydrocarbon-based compounds and/or silicone compounds. In the case of a mixture of different pasty fatty substances, the hydrocarbon-based pasty compounds (containing mainly hydrogen and carbon atoms and optionally ester groups) are preferably used in major proportion.

Among the pasty compounds which may be used in the composition according to the invention, mention may be made of lanolins and lanolin derivatives such a's acetylated lanolins or oxypropylenated lanolins or isopropyl lanolate, having a viscosity of from 18 to 21 Pa·s, preferably 19 to 20.5 Pa·s, and/or a melting point of from 30 to 55° C., and mixtures thereof. It is also possible to use esters of fatty acids or of fatty alcohols, in particular those containing from 20 to 65 carbon atoms (melting point of about from 20 to 35° C. and/or viscosity at 40° C. ranging from 0.1 to 40 Pa·s), such as triisostearyl or cetyl citrate; arachidyl propionate; polyvinyl laurate; cholesterol esters, such as triglycerides of plant origin, such as hydrogenated plant oils, viscous polyesters such as poly(12-hydroxystearic acid), and mixtures thereof.

Mention may also be made of pasty silicone fatty substances such as polydimethylsiloxanes (PDMSs) containing pendent chains of the alkyl or alkoxy type containing from 8 to 24 carbon atoms, and having a melting point of 20-55° C., such as stearyldimethicones, in particular those sold by Dow-Corning under the trade names DC2503 and OC25514, and mixtures thereof.

The pasty fatty substance may be present in the composition according to the invention in a proportion of from 0.01% to 60% by weight, relative to the total weight of the composition, preferably ranging from 0.5% to 45% by weight, and better still ranging from 2% to 30% by weight, in the composition.

The composition according to the invention can contain emulsifying surfactants, present in particular in a proportion ranging from 2% to 30% by weight relative to the total weight of the composition, and better still from 5% to 15%. These surfactants may be chosen from anionic and nonionic surfactants. Reference may be mad to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of the said reference, for the anionic and nonionic surfactants.

The surfactants preferably used in the composition according to the invention are chosen from:
nonionic surfactants: fatty acids, fatty alcohols, polyethoxylated or polyglycerolated fatty alcohols such as poly-ethoxylated stearyl or cetylstearyl alcohol, fatty acid esters of sucrose, alkylglucose esters, in particular polyoxyethylenated fatty esters of C₁-C₆ alkyl glucose, and mixtures thereof;
anionic surfactants: C₁₆-C₃₀ fatty acids neutralized with amines, aqueous ammonia or alkaline salts, and mixtures thereof.

Surfactants that make it possible to obtain an oil-in-water or wax-in-water emulsion are preferably used.

The composition according to the invention may comprise, in addition to the block polymer and the semicrystalline polymer described above, an additional polymer such as a film-forming polymer.

The additional film-forming polymer may be present in the composition according to the invention in a dry matter content ranging from 0.1% to 60% by weight, preferably from 0.5% to 40% by weight and better still from 1% to 30% by weight relative to the total weight of the composition.

Preferably, the additional film-forming polymer does not comprise crystallizable units. Were it to contain crystallizable units, these would represent less than 30% by weight of the total weight of the polymer.

Among the film-forming polymers that may be used in the composition of the present invention, mention may be made of synthetic polymers, of radical-mediated type or of polycondensate type, and polymers of natural origin, and mixtures thereof.

The expression “radical-mediated film-forming polymer” means a polymer obtained by polymerization of monomers containing unsaturation, in particular ethylenic unsaturation, each monomer being capable of homopolymerizing (unlike polycondensates).

The film-forming polymers of radical-mediated type may be, in particular, vinyl polymers or copolymers, in particular acrylic polymers.

The vinyl film-forming polymers can result from the polymerization of monomers containing ethylenic unsaturation and containing at least one acidic group and/or esters of these acidic monomers and/or amides of these acidic monomers.

Monomers bearing an acidic group which may be used are α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), specially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl(meth) acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates that may be mentioned are hydroxyethyl acrylate, 2-hydroxypropyl acrylate, hydroxyethyl methacrylate and 2-hydroxypropyl methacrylate.

Among the aryl(meth)acrylates that may be mentioned are benzyl acrylate and phenyl acrylate.

The (meth)acrylic acid esters that are particularly preferred are the alkyl(meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth) acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or colypolymerization of monomers chosen from vinyl esters and styrene monomers. In particular, these monomers may be polymerized with acid monomers and/or esters thereof and/or amides thereof, such as those mentioned above.

Examples of vinyl esters that may be mentioned are vinyl acetate, vinyl neo-decanoate, vinyl pivalate, vinyl benzoate and vinyl t-butylbenzoate.

Styrene monomers that may be mentioned are styrene and α-methylstyrene.

Among the film-forming polycondensates that may be mentioned are polyurethanes, polyesters, polyesteramides, polyamides, epoxyester resins and polyureas.

The polyurethanes may be chosen from anionic, cationic, nonionic and amphoteric polyurethanes, polyurethane-acrylics, polyurethane-polyvinylpyrrolidones, polyester-polyurethanes, polyether-polyurethanes, polyureas and polyurea/polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulphonylbiphenyl and methylenebiphenyl nuclei. As examples of difunctional aromatic monomers also bearing a group —SO₃M, mention may be made of: sulphoisophthalic acid, sulphoterephthalic acid, sulphophthalic acid, 4-sulphonaphthalene-2,7-dicarboxylic acid.

The copolymers preferably used are those based on isophthalate/sulphoisophthalate, and more particularly copolymers obtained by condensation of diethylene glycol, cyclohexanedimethanol, isophthalic acid and sulphoisophthalic acid.

The polymers of natural origin, optionally modified, may be chosen from shellac resin, sandarac gum, dammar resins, elemi gums, copal resins and cellulose polymers, and mixtures thereof.

According to a first embodiment of the composition according to the invention, the film-forming polymer may be a water-soluble polymer and may be present in an aqueous phase of the composition; the polymer is thus solubilized in the aqueous phase of the composition. Examples of water-soluble film-forming polymers that may be mentioned are:

proteins, for instance proteins of plant origin such as wheat proteins and soybean proteins; proteins of animal origin such as keratins, for example keratin hydrolysates and sulphonic keratins;

polymers of cellulose such as hydroxyethylcellulose, hydroxypropylcellulose, methylcellulose, ethylhydroxyethylcellulose and carboxymethylcellulose, and quaternized cellulose Derivatives;

acrylic polymers or copolymers, such as polyacrylates or polymethacrylates;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, which are optionally modified, such as:

gum arabics, guar gum, xanthan derivatives, karaya gum;

alginates and carrageenans;

glycosaminoglycans, hyaluronic acid and derivatives thereof;

shellac resin, sandarac gum, dammar resins, elemi gums and copal resins;

deoxyribonucleic acid;

mucopolysaccharides such as chondroitin sulphate, and mixtures thereof.

According to another embodiment of the composition according to the invention, the film-forming polymer may be a polymer dissolved in a liquid fatty phase comprising organic solvents or oils such as those described above (the film-forming polymer is thus said to be a liposoluble polymer). For the purposes of the invention, the expression “liquid fatty phase” means a fatty phase which is liquid at room temperature (25° C.) and atmospheric pressure (760 mmHg, i.e. 10⁵ Pa), composed of one or more fatty substances that are liquid at room temperature, such as the oils described above, which are generally mutually compatible.

The liquid fatty phase preferably comprises a volatile oil, optionally mired with a non-volatile oil, the oils possibly being chosen from those mentioned above.

Examples of liposoluble polymers which may be mentioned are copolymers of vinyl ester (the Vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl/ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based-radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers which may be mentioned are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Examples of liposoluble film-forming polymers which may also be mentioned are liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from copolymers of polyvinyl stearate, polyvinyl stearate crosslinked with the aid of divinylbenzene, of diallyl ether or of diallyl phthalate, polystearyl (meth)acrylate, polyvinyl laurate and polylauryl (meth)acrylate, it being possible for these poly(meth)acrylates to be crosslinked with the aid of ethylene glycol dimethacrylate or tetraethylene glycol dimethacrylate.

The liposoluble copolymers defined above are known and are described in particular in patient application FR-A-2 232 303; they may have a weight-average molecular weight ranging from 2 000 to 500 000 and preferably from 4 000 to 200 000.

As liposoluble film-forming polymers which may be used in the invention, mention may also be made of polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

The film-forming polymer may also be present in the composition in the form of particles dispersed in an aqueous phase or in a non-aqueous solvent phrase, which is generally known as a latex or pseudolatex. The techniques for preparing these dispersions are well known to those skilled in the art.

Aqueous dispersions of film-forming polymers which may be used are the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ by the company Daito Kasey Kogyo; Syntran 5760 by the company Interpolymer or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulphopolyesters sold under the brand name “Eastman AQ®” by the company Eastman Chemical Products, vinyl dispersions, for instance “Mexomer PAM” and also acrylic dispersions in isododecane, for instance “Mexomer PAP” by the company Chimex.

According to one embodiment, the composition according to the invention advantageously comprises a film-forming linear ethylenic block polymer as described above and particles of film-forming polymer in dispersion in an aqueous phase.

The composition according to the invention may comprise a plasticizer, which promotes the formation of a film with the film-forming polymer. Such a plasticizer may be chosen from any of the compounds known to those skilled in the art as being capable of satisfying the desired function.

### Additives

The composition according to the invention may also comprise a dyestuff, for instance pulverulent dyestuffs, liposoluble dyes and water-soluble dyes. This dyestuff may be present in a content ranging from 0.01% to 30% by weight relative to the total weight of the composition.

The pulverulent dyestuffs may be chosen from pigments and nacres.

The pigments may be white or coloured, mineral and/or organic, and coated or uncoated. Among the mineral pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the abovementioned type, and nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice, methylene blue, the disodium salt of ponceau, the disodium salt of alizarin green, quinoline yellow, the trisodium salt of amaranthus, the disodium salt of tartrazine, the monosodium salt of rhodamine, the disodium-salt of fuchsin, and xanthophyll.

The fillers may be chosen from those that are well known to a person skilled in the art and commonly used in cosmetic compositions. The fillers may be mineral or organic and lamellar or spherical. Mention may be made of talc, mica, silica, kaolin, polyamide powder for instance Nylon® (Orgasol from Atochem), poly-β-alanine powder and polyethylene powder, tetra fluoroethylene polymer powders for instance Teflon®, lauroyllysine, starch, boron nitride, expand hollow polymer microspheres such as those made of polyvinylidene chloride/acrylonitrile, for instance Expancel® (Nobel Industrie), acrylic powders such as Polytrap® (Dow Corning), polymethyl methacrylate particles and silicone resin microbeads (for example Tospearls® from Toshiba), precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The fillers may represent from 0.1% to 25% and better still from 1% to 20% by weight relative to the total weight of the composition.

The composition of the invention may additionally comprise any additive commonly used in cosmetics, such as antioxidants, preservatives, fragrances, neutralizing agents, gelling agents, thickeners, vitamins and mixtures thereof.

The gelling agents that may be used in the compositions according to the invention may be organic or mineral, and polymeric or molecular, hydrophilic or lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name “Bentone 38V®” by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:
trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references “Aerosil R812®” by the company Degussa, and “Cab-O-Sil TS-530®” by the company Cabot;
dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTFA (6th edition, 1995). They are sold, for example, under the references “Aerosil R972®” and “Aerosil R974®” by the company Degussa, and “Cab-O-Sil TS-610®” and “Cab-O-Sil TS-720®” by the company Cabot.

The hydrophobic fumed silica particularly has a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names “KSG6®”, “KSG16®” and “KSG18®” from Shin Etsu, “Trefil E-505C®” and “Trefil E-506C®” from Dow Corning, “Gransil SR-CYC®”, “SR DMF 10®”, “SR-DC556®”, “SR 5CYC Gel®”, “SR DMF 10 Gel®” and “SR DC 556 Gel®” from Grant Industries and “SF 1204®” and “JK 113®” from General Electric; ethylcellulose, for instance that sold under the name “Ethocel®” by Dow Chemical and galactomannans comprising from one to six and in particular from two to four hydroxyl groups per monosaccharide, substituted with a saturated or unsaturated alkyl chain, for instance guar gum alkylated with C₁ to C₆, and in particular C₁ to C₃, alkyl chains, and mixtures thereof. The “diblock” or “triblock” type block copolymers of the polystyrene/polyisoprene or polystyrene/polybutadiene type such as those sold under the name “Luvitol HSB®” by the company BASF, of the polystyrene/copoly(ethylene-propylene) type such as those sold under the name “Kraton®” by the company Shell Chemical Co or of the polystyrene/copoly(ethylene-butylene) type.

Among the lipophilic gelling agents which may be used in the compositions according to the invention, mention may also be made of fatty acid esters of dextrin such as dextrin palmitates, especially such as those sold under the names “Rheopearl TL®” or “Rheopearl KL®” by the company Chiba Flour.

The composition according to the invention advantageously has a dry matter content of greater than or equal to 45%, preferably of greater than or equal to 46%, better still of greater than or equal to 47%, even better still of greater than or equal to 48%, preferably still of greater than or equal to 50%, better still of greater than or equal to 55%, possibly ranging up to 60%.

### Protocol for Measuring the Dry Matter Content or Dry Extract

The dry matter content, i.e. the non-volatile matter content, may be measured in different ways, mention may be made for example of the methods of drying in an oven, the methods of drying by exposure to infrared radiation and chemical methods by titration of water according to Karl Fischer.

Preferably, the dry extract of the compositions according to the invention is measured on a Mettler Toledo HG 53 balance (Halogen Moisture Analyzer).

A mascara sample (2-3 g) is deposited in an aluminium dish and subjected to a temperature of 120° C. for 60 minutes. The measurement of the dry extract corresponds to the monitoring of the mass of the sample as a function of time. The final solids content is therefore the percentage of the final mass (after 60 min) relative to the initial mass: DE=final mass/initial mass)×100.

Needless to say, a person skilled in the art will take care to select the optional additional additives and/or the amount thereof such that the advantageous properties of the composition according to the invention are not, or are not substantially, adversely affected by the addition envisaged.

The composition according to the invention may be manufactured by known processes generally used in the cosmetic field.

Preferably, the composition according to the invention is a mascara.

The composition according to the invention may be packaged in a cosmetic set comprising a container delimiting at least one compartment which comprises the said composition, the said container being closed by a closing member.

The container is preferably combined with an applicator, especially in the form of a brush comprising an arrangement of bristles maintained by a twisted wire. Such a twisted brush is described especially in U.S. Pat. No. 4,867,622. It may also be in the form of a comb comprising a plurality of application members, obtained especially by moulding. Such combs are described for example in patent FR 2 796 529. The applicator may be integrally attached to the container, as described for example in patent FR 2 761 959. Advantageously, the applicator is integrally attached to a rod which is itself integrally attached to the closing-member.

The closing member may be coupled to the container by screwing. Alternatively, the coupling between the closing member and the container is done other than by screwing, especially via a bayonet mechanism, by click-fastening or by tightening. The term “click-fastening” in particular means any system involving the crossing of a bead or cord of material by elastic deformation of a portion, especially the closing member, followed by return to the elastically unconstrained position of the said portion after the crossing of the bread or cord.

The container may be at least partially made of thermoplastic material. Examples of thermoplastic materials that may be mentioned include polypropylene or polyethylene.

Alternatively, the container is made of non thermoplastic material, especially glass or metal (or alloy).

The container is preferably equipped with a drainer arranged in the region of the aperture of the container. Such a drainer makes it possible to wipe the applicator and possibly the rod to which it may be integrally attached. Such a drainer is described for example in patent FR 2 792 618.

The content of the patents or patent applications cited above are incorporated by reference into the present application.

The invention is illustrated in greater detail in the following examples.

The quantities are given in grams.

### EXAMPLE 1

### Preparation of a poly(isobornyl acrylate/isobutyl methacrylate/2-ethylhexyl acrylate)polymer

100 g of isododecane are introduced into a 1 litre reactor, and then the temperature is increased so as to pass from room temperature (25° C.) to 90° in 1 hour.

120 g of isobornyl acrylate, 90 g of isobutyl methacrylate, 110 g of isododecane and 1.8 g of 2,5-bis(2-ethylhexanoylperoxy)-2,5-dimethylhexane (Trigonox® 141 from Akzo Nobel) are then added at 90° C. and over 1 hour.

The mixture is maintained for 1 h 30 min at 90° C.

90 g of 2-ethylhexyl acrylate, 90 g of isododecane and 1.2 g of 2,5-bis(2-ethylhexanoylperoxy)-2,5-dimethylhexane are then introduced into the preceding mixture, still at 90° C. and over 30 minutes.

The mixture is maintained for 3 hours at 90° C., and then the whole is cooled.

A solution containing 50% polymer active material in isododecane is obtained.

A polymer comprising a poly(isobornyl acrylate/isobutyl methacrylate) first block with a Tg of 80° C., a poly(2-ethylhexyl acrylate) second block with a Tg of −70° C. and an intermediate black which is an isobornyl acrylate/isobutyl methacrylate/2-ethylhexyl acrylate random polymer is obtain.

This polymer has a weight-average mass of 77 000 g/Mol and a number-average mass of 19 000, i.e. a polydispersity index I of 4.05.

### EXAMPLE 2

### Preparation of a poly(isobornyl acrylate/isobornyl methacrylate/2-ethylhexyl acrylate)polymer

100 g of isododecane are introduced into a 1 litre reactor, and then the temperature is increased so as to pass from room temperature (25° C.) to 90° C. in 1 hour.

105 g of isobornyl acrylate, 105 g of isobornyl methacrylate, 110 g of isodecane and 1.8 g of 2,5-bis(2-ethylhexanoylperoxy)-2,5-dimethylhexane (Trigonox® 141 from Akzo Nobel) are then added at 90° C. and over 1 hour.

The mixture is maintained for 1 h 30 min at 90° C.

90 g of 2-ethylhexyl acrylate, 90 g of isodo-decane and 1.2 g of 2,5-bis(2-ethylhexanoylperoxy)-2,5-dimethylhexane are then introduced into the preceding mixture, still at 90° C. and over 30 minutes.

The mixture is maintained for 3 hours at 90° C., and then the whole is cooled.

A solution containing 50% polymer active material in isododecane is obtained.

A polymer comprising a poly(isobornyl acrylate/isobornyl methacrylate) first block with a Tg of 110° C., a poly(2-ethylhexyl acrylate) second block with a Tg of −70° C. and an intermediate block which is an isobornyl acrylate/isobornyl methacrylate/2-ethylhexyl acrylate random polymer is obtained.

This polymer has a weight-average mass of 103 900 g/Mol and a number-average mass of 21 300, i.e. a polydispersity index I of 4.89.

### EXAMPLE 3

### Preparation of a Poly(Isobornyl methacrylate/isobutyl methacrylate/isobutyl acrylate)polymer

100 g of isododecane are introduced into a 1 litre reactor, and then the temperature is increased so as to pass from room temperature (25° C.) to 90° C. in 1 hour.

120 g of isobornyl methacrylate, 90 g of isobutyl methacrylate, 110 g of isododecane and 1.8 g of 2,5-bis(2-ethylhexanoylperoxy)-2,5-dimethylhexane (Trigonox® 141 from Akzo Nobel) are then added at 90° C. and over 1 hour.

The mixture is maintained for 1 h 30 min at 90° C.

90 g of isobutyl acrylate, 90 g of isodo-decane and 1.2 g of 2,5-bis(2-ethylhexanoylperoxy)-2,5-dimethylhexane are then introduced into the preceding mixture, still at 90° C. and over 3.0 minutes.

The mixture is maintained for 3 hours at 90° C., and then the whole is cooled.

A solution containing 50% polymer active material in isododecane is obtained.

A polymer comprising a poly-(isobornyl methacrylate/isobutyl methacrylate) first block with a Tg of 95° C., a poly(isobutyl acrylate) second block with a Tg of −20° C. and an intermediate block which is an isobornyl methacrylate/isobutyl methacrylate/isobutyl acrylate random polymer is obtained.

This polymer has a weight-average mass of 100 700 g/Mol and a number-average mass of 20 800, i.e. a polydispersity index I of 4.85.

### EXAMPLE 4

### Preparation of a poly(isobornyl acrylate/isobutyl methacrylate/isobutyl acrylate)polymer

100 g of isododecane are introduced into a 1 litre reactor, and then the temperature is increased so as to pass from room temperature (25° C.) to 90° C. in 1 hour.

120 g of isobornyl acrylate, 90 g of isobutyl methacrylate, 110 g of isododecane and 1.6 g of 2,5-bis(2-ethylhexanoylperoxy)-2,5-dimethylhexane (Trigonox® 141 from Akzo Nobel) are then added at 90° C. and over 1 hour.

The mixture is maintained for 1 h 30 min at 90° C.

90 g of isobutyl acrylate, 90 g of isodo-decane and 1.2 g of 2,5-bis(2-ethylhekanoylperoxy)-2,5-dimethylhexane are then introduced into the preceding mixture, still at 90° C. and over 30 minutes.

The mixture is maintained for 3 hours at 90° C., and then the whole is cooled.

A solution containing 50% polymer active material in isododecane is obtained.

A polymer comprising a poly(isobornyl acrylate/isobutyl methacrylate) first block with a Tg of 75° C., a polyisobutyl acrylate) second block with a Tg of −20° C. and an intermediate block which is an isobornyl acrylate/isobutyl methacrylate/isobutyl acrylate random polymer is obtained.

This polymer has a weight-average mass of 144 200 g/Mol and a number-average mass of 49 300, i.e. a polydispersity index I of 2.93.

The following polymer may be prepared.

### EXAMPLE 5

### Preparation of a poly(isobornyl acrylate/isobutyl methacrylate/2-ethylhexyl acrylate)polymer

100 g of isododecane are introduced into a 1 litre reactor, and then the temperature is increased so as to pass from room temperature (25° C.) to 90° C. in 1 hour.

54 g of isobornyl acrylate, 75.6 g of isobutyl methacrylate, 50.4 g of 2-ethylhexyl acrylate, 110 g of isododecane and 1.8 g of 2,5-bis(2-ethyl-hexanoylperoxy)-2,5-dimethylhexane (Trigenox® 141 from Akzo Nobel) are then added at 90° C. and over 1 hour.

The mixture is maintained for 1 h 30 min at 90° C.

120 g of 2-ethylhexyl acrylate, 90 g of isododecane and 1.2 g of 2,5-bis(2-ethylhexanoyl-peroxy)-2,5-dimethylhexane are then introduced into the preceding mixture, still at 90° C. and over 1 hour.

The mixture is maintained for 3 hours at 90° C., and then the whole is cooled.

A solution containing 50% polymer active material in isododecane is obtained.

A polymer-comprising a poly(isobornyl acrylate/isobutyl methacrylate/2-ethylhexyl acrylate) first block with a Tg of 25° C., a poly(2-ethylhexyl acrylate) second block with a Tg of −50° C. and an intermediate block which is an isobornyl acrylate/isobutyl methacrylate/2-ethylhexyl acrylate random polymer is obtained.

### EXAMPLES 6 TO 10

### Wax-Free Mascaras

The following mascaras comprising a semicrystalline polymer and a block polymer according to the invention (Examples 7 to 10) and according to the prior art (Example 6) were prepared:

| | | Example 7 | Example 8 | Example 9 | Example 10 |
|---|---|---|---|---|---|
| | Example 6 | (according to | (according to | (according to | (according to |
| | (comparative) | the invention) | the invention) | the invention) | the invention) |
| Polystearyl acrylate | 23.3 | 23.3 | 23.3 | 15 | 8.5 |
| Block polymer of Example 3 | — | — | 11.6 | — | — |
| (as AM*) | | | | | |
| Block polymer of Example 4 | — | 12.5 | — | 16 | 20 |
| (as AM) | | | | | |
| Polyisobutene | 11.6 | — | — | — | — |
| Isododecane | — | 12.5 | 11.6 | 16 | 20 |
| Stearic acid | 5.8 | 5.8 | 5.8 | 5.8 | 5.8 |
| Triethanolamine | 2.4 | 2.4 | 2.4 | 2.4 | 2.4 |
| Aminoethylpropanediol | 0.5 | 0.5 | 0.5 | 0.5 | 0.5 |
| Hydroxyethylcellulose | 0.9 | 0.9 | 0.9 | 0.9 | 0.9 |
| Gum arabic | 3.45 | 3.45 | 3.45 | 3.45 | 3.45 |
| Black iron oxide | 8 | 8 | 8 | 8 | 8 |
| Water | qsp 100 | qsp 100 | qsp 100 | qsp 100 | qsp 100 |

*AM: active material

For each composition, the dry extract its determined according to the method indicated above, the charge in vitro and the staying power.

The charge in vitro is measured by gravimetry on specimens of curled Caucasian hair (30 hair strands 1 cm long spread over a distance of 1 cm).

The specimen is made up by carrying out 3×10 passages of mascara 2 minutes apart with collection of product between reach series of 10.

The specimen is dried for 10 min at room temperature and then weighed.

This measurement is performed on 6 specimens.

The charge is in fact the quantity of material deposited on the specimen=mass of specimen made up−bare specimen mass.

The mean charge is the mean of the measurements carried out on the 6 specimens.

The staying power of the film formed by the composition according to the invention is evaluated by measuring the water resistance, as a function of time, of a film of composition spread onto a glass plate and subjected to stirring in aqueous medium. The protocol is as follows:

At ambient temperature (25° C.), a layer of composition 300 μm thick (before drying) with a surface area of 9 cm×9 cm is spread onto a glass plate with a surface area of 10 cm×10 cm, and is then left to dry for 24 hours at 30° C. and 50% relative humidity. After drying, the plate is placed in a 2 litre crystallizing dish 19 cm in diameter, filled with 1 little of water and placed on a heating magnetic stirrer sold under the name RCT basic by the company IKA Labortechnik. A smooth cylindrical PTFE magnetic bar (6 cm long; 1 cm diameter) is then placed on the film. The stirring speed is set to position 5. The water temperature is controlled using a thermometer to a temperature of 20° C. or 40° C. At time t₀=0, the stirring is started. The time t (expressed in minutes) after which the film begins to detach or debond from the plate or when a hole the size of the stirring magnetic bar is observed, i.e. when the hole has a diameter of 6 cm, is measured. The water resistance of the film, corresponds to the time t measured.

The results which follow were obtained.

| | Example | Example | Example | Example | Example |
|---|---|---|---|---|---|
| | 6 | 7 | 8 | 9 | 10 |
| Dry extract | 52.9 | 55.8 | 55.4 | 51.9 | 46.5 |
| measured in | | | | | |
| (%) | | | | | |
| Charge in | 10.42 ± 1.4 | 16.73 ± 0.9 | 12.27 ± 1.62 | 17.3 ± 2.8 | 13.08 ± 1.3 |
| vitro (Mg) | | | | | |
| Staying | 24 sec. | 1 min and | About 1 | 7 min and | 4 min and |
| power | | 49 sec. | min | 54 sec | 52 sec |

It is observed that the mascaras of Examples 7 to 10 according to the invention have a greater staying power than the mascara containing no block polymer (Example 8), and a higher change in vitro.

These mascaras, after application to the eyelashes, make it possible to obtain a glossy fi-m, good staying power and a volumizing effect.

### EXAMPLE 11

### Mascara

The following mascara may be prepared:

| | |
|---|---|
| Block polymer of Example 4 | 10 a.m. |
| Isododecane | 10 |
| Semicrystalline polymer | 10 |
| (polystearyl acrylate) | |
| Beeswax | 10 |
| Stearic acid | 5.8 |
| Aminomethylpropanediol | 0.5 |
| Black iron oxide | 8.0 |
| Hydroxyethylcellulose | 0.9 |
| Gum arabic | 3.45 |
| Triethanolamine | 2.4 |
| Water | qs 100 |

### EXAMPLE 12

### Mascara

The following mascara may be prepared:

| | |
|---|---|
| Block polymer of Example 4 | 15 a.m. |
| Isododecane | 15 |
| Semicrystalline polymer | 8 |
| (polystearyl acrylate) | |
| Candelilla wax | 5 |
| Paraffin wax | 4 |
| Stearic acid | 5.8 |
| Aminomethylpropanediol | 0.5 |
| Black iron oxide | 8.0 |
| Hydroxyethylcellulose | 0.9 |
| Gum arabic | 3.45 |
| Triethanolamine | 2.4 |
| Water | qs 100 |

1. Composition for coating keratin fibres comprising a cosmetically acceptable organic liquid medium, at least one film-forming linear ethylenic block polymer, and at least one semicrystalline polymer.

2. Composition according to claim 1, characterized in that the said block polymer is free of styrene.

3. Composition according to claim 1 or 2, characterized in that the said block polymer is non-elastomeric.

4. Composition according to one of the preceding claims, characterized in that the block polymer comprises at least one first block and at least one second block having different glass transition temperatures (Tg), the said first and second blocks being linked together via an intermediate block comprising at least one constituent monomer of the first block and at least one constituent monomer of the second block.

5. Composition according to the preceding claim, characterized in that the first block and second blocks of the block polymer are mutually incompatible.

6. Composition according to the preceding claim, characterized in that the first block of the block polymer is chosen from:
a) a block with a Tg of greater than or equal to 40° C.,
b) a block with a Tg of less than or equal to 20° C.,
c) a block with a Tg of between 20 and 40° C., and
the second block is chosen from α-category a), b) or c) different from the first block.

7. Composition according to claim 6, characterized in that the block of the block polymer with a Tg of greater than or equal to 40° C. is totally or partially derived from one or more monomers, which are such that the homopolymer prepared from these monomers has a glass transition temperature of greater than or equal to 40° C.

8. Composition according to the preceding claim, characterized in that the monomers whose corresponding homopolymer has a glass transition temperature of greater than or equal to 40° C. are chosen from the following monomers:
methacrylates of formula CH₂═C(CH₃)—COOR₁ in which R₁ represents a linear or branched unsubstituted alkyl group containing from 1 to 4 carbon atoms, such as a methyl, ethyl, propyl or isobutyl group or R₁ represents a C₄ to C₁₂ cycloalkyl group,
acrylates of formula CH₂═CH—COOR₂ in which R₂ represents a C₄ to C₁₂ cycloalkyl group such as isobornyl acrylate or a tert-butyl group,
(meth)acrylamides of formula: 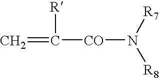
in which R₇ and R₈, which may be identical or different, each represent a hydrogen atom or a linear or branched alkyl group of 1 to 12 carbon atoms such as an n-butyl, t-butyl, isopropyl, isohexyl, isooctyl or isononyl group; or R₇ represents H and R₈ represents a 1,1-dimethyl-3-oxobutyl group, and R′ denotes H or methyl,
and mixtures thereof.

9. Composition according to claim 7 or 8, characterized in that the monomers whose corresponding homopolymer has a glass transition temperature of greater than or equal to 40° C. are chosen from methyl methacrylate, isobutyl methacrylate and isobornyl (meth)acrylate, and mixtures thereof.

10. Composition according to claim 6, characterized in that the block of the block polymer with a Tg of less than or equal to 20° C. is totally or partially derived from one or more monomers which are such that the homopolymer prepared from these monomers has a glass transition temperature of less than or equal to 20° C.

11. Composition according the preceding claim, characterized in that the monomers whose corresponding homopolymer has a glass transition temperature of less than or equal to 26° C. are chosen from the following monomers:
acrylates of formula CH₂═CHCOOR₃, R₃ representing a linear or branched C₁ to C₁₂ unsubstituted alkyl group, with the exception of the tert-butyl group, in which one or more hetero atoms chosen from O, N and S is (are) optionally intercalated,
methacrylates of formula CH₂═C(CH₃)—COOR₄, R₄ representing a linear or branched C₆ to C₁₂ unsubstituted alkyl group, in which one or more hetero atoms chosen from O, N and S is (are) optionally intercalated,
vinyl esters of formula R₅—CO—O—CH═CH₂ in which R₅ represents a linear or branched C₄ to C₁₂ alkyl group,
C₄ to C₁₂ alkyl vinyl ethers,
N— (C₄ to C₁₂)alkyl acrylamides, such as N-octylacrylamide,
and mixtures thereof.

12. Composition according to claim 10 or 11, characterized in that the monomers whose corresponding homopolymer has a glass transition temperature of less than or equal to 20° C. are chosen from alkyl acrylates whose alkyl chain contains from 1 to 10 carbon atoms, with the exception of the tert-butyl group.

13. Composition according to claim 6, characterized in that the block with a Tg of between 20 and 40° C. is totally or partially derived from one or more monomers which are such that the homopolymer prepared from these monomers has a glass transition temperature of between 20 and 40° C.

14. Composition according to claim 6, characterized in that the block with a Tg of between 20 and 40° C. is totally or partially derived from monomers which are such that the corresponding homopolymer has a Tg of greater than or equal to 40° C. and from monomers which are such that the corresponding homopolymer has a Tg of less than or equal to 20° C.

15. Composition according to claim 13 or 14, characterized in that the block with a Tg of between 20 and 40° C. is totally or partially derived from monomers chosen from methyl methacrylate, isobornyl acrylate and methacrylate, trifluoroethyl methacrylate, butyl acrylate and 2-ethylhexyl acrylate, and mixtures thereof.

16. Composition according to one of claims 1 to 6, characterized in that the block polymer comprises at least one first block and at least one second block, the first block having a glass transition temperature (Tg) of greater than or equal to 40° C. and the second block having a glass transition temperature of less than or equal to 20° C., the said first and second blocks being linked together via an intermediate block comprising at least one constituent monomer of the first block and at least one-constituent monomer of the second block.

17. Composition according to the preceding claim, characterized in that the first block of the block polymer is totally or partially derived from one or more monomers which are such that the homopolymer prepared from these monomers has a glass transition temperature of greater than or equal to 40° C.

18. Composition according to claim 16, characterized in that the first block of the block polymer is a copolymer derived from monomers which are such that the homopolymer prepared from these monomers has a glass transition temperature of greater than or equal to 40° C.

19. Composition according to claim 17 or 18, characterized in that the monomers whose corresponding homopolymer has a glass transition temperature of greater than or equal to 40° C. are chosen from the following monomers:
methacrylates of formula CH₂═C(CH₃)—COOR₁ in which R₁ represents a linear or branched unsubstituted alkyl group containing from 1 to 4 carbon atoms, such as a methyl, ethyl, propyl or isobutyl group or R₁ represents a C₄ to C₁₂ cycloalkyl group,
acrylates of formula CH₂═CH—COOR₂ in which R₂ represents a C₄ to C₁₂ cycloalkyl group such as isobornyl acrylate or a tert-butyl group,
(meth)acrylamides of formula: 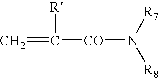
in which R₇ and R₅, which may be identical or different, each represent a hydrogen atom or a linear or branched alkyl group of 1 to 12 carbon atoms such as an n-butyl, t-butyl, isopropyl, isohexyl, isooctyl or isononyl group; or R₇ represents H and R₈ represents a 1,1-dimethyl-3-oxobutyl group, and R′ denotes H or methyl
and mixtures thereof.

20. Composition according to one of claims 17 to 19, characterized in that the monomers whose corresponding homopolymer has a glass transition temperature of greater than or equal to 40° C. are chosen from methyl methacrylate, isobutyl methacrylate and isobornyl(meth)acrylate, and mixtures thereof.

21. Composition according to one of claims 16 to 20, characterized in that the proportion of the first block having a Tg of greater than or equal to 40° C. of the block polymer ranges from 20% to 90% by weight, better still from 30% to 80% and even better still from 50% to 70% by weight of the polymer.

22. Composition according to one of claims 16 to 21, characterized in that the second block of the block polymer is totally or partially derived from one or more monomers which are such that the homopolymer prepared from these monomers has a glass transition temperature of less than or equal to 2-0° C.

23. Composition according to one of claims 16 to 22, characterized in that the second block of the block polymer is a homopolymer derived from monomers which are such that the homopolymer prepared from these monomers has a glass transition temperature of less than or equal to 20° C.

24. Composition according to claim 22 or 23, characterized in that the monomers whose corresponding homopolymer has a glass transition temperature of less than or equal to 20° C. are chosen from the following monomers:
acrylates of formula CH₂═CHCOOR₃, R₃ representing a linear or branched C₁ to C₁₂ unsubstituted alkyl group, with the exception of the tert-butyl group, in which one or more hetero atoms chosen from O, N and S is (are) optionally intercalated,
methacrylates of formula CH₂═C(CH₃)—COOR₄, R₄ representing a linear or branched C₆ to C₁₂ unsubstituted alkyl group, in which one or more hetero atoms chosen from O, N and S is (are) optionally intercalated,
vinyl esters of formula R₅—CO—O—CH═CH₂ in which R₅ represents a linear or branched C₄ to C₁₂ alkyl group,
(—C₄ to C₁₂ alkyl vinyl ethers,
N—(C₄ to C₁₂) alkyl acrylamides, such as N-octylacrylamide,
and mixtures thereof.

25. Composition according to one of claims 22 to 24, characterized in that the monomers whose corresponding homopolymer has a glass transition temperature of less than or equal to 20° C. are chosen from alkyl acrylates whose alkyl chain-contains from 1 to 10 carbon atoms, with the exception of the butyl group.

26. Composition according to one of claims 16 to 25, characterized in that the proportion of the second block with a Tg of less than or equal to 20° C. of the block polymer ranges from 5% to 75% by weight, better still from 15% to 50% and even better still from 25% to 45% by weight of the polymer.

27. Composition according to one of claims 1 to 6, characterized in that the block polymer comprises at least one first block and at least one second block, the first block having a glass transition temperature (Tg) of between 20 and 40° C. and the second block having a glass transition temperature of less than or equal to 20° C. or a glass transition temperature of greater than or equal to 40° C., the said first and second blocks being linked together via an intermediate block comprising at least one constituent monomer of the first block and at least one constituent monomer of the second block.

28. Polymer according to the preceding claim, characterized in that the first block with a Tg of between 20 and 40° C. of the block polymer is totally or partially derived from one or more monomers which are such that the homopolymer prepared from these monomers has a glass transition temperature of between 20 and 40° C.

29. Composition according to claim 27 or 28, characterized in that the first block with a Tg of between 20 and 40° C. of the block polymer is a copolymer derived from monomers which are such that the corresponding homopolymer has a Tg of greater than or equal to 40° C. and from monomers which are such that the corresponding homopolymer has a Tg of less than or equal to 20° C.

30. Composition according to one of claims 27 to 29, characterized in that the first block with a Tg of between 20 and 40° C. of the block polymer is derived from monomers chosen from methyl methacrylate, isobornyl acrylate and methacrylate, butyl acrylate and 2-ethylhexyl acrylate, and mixtures thereof.

31. Composition according to one of claims 27 to 30, characterized in that the proportion of the first block with a Tg of between 2.0 and 40° C. ranges from 10% to 85%, better still from 30% to 60% and even better still from 50% to 70% by weight of the polymer.

32. Composition according to any one of claims 27 to 31, characterized in that the second block of the block polymer has a Tg of greater than or equal to 40° C. and is totally or partially derived from one or more monomers which are such that the homopolymer prepared from these monomers has a glass transition temperature of greater than or equal to 40° C.

33. Composition according to any one of claims 27 to 32, characterized in that the second block of the block polymer has a Tg of greater than or equal to 40° C. and is a homopolymer derived from monomers which are such that the homopolymer prepared from these monomers has a glass transition temperature of greater than or equal to 40° C.

34. Composition according to claim 32 or 33, characterized in that the monomers whose corresponding homopolymer has a glass transition temperature of greater than or equal to 40° C. are chosen from the following monomers:
methacrylates of formula CH₂═C(CH₃)—COOR₁ in which R₁ represents a linear or branched unsubstituted alkyl group containing from 1 to 4 carbon atoms, such as a methyl, ethyl, propyl or isobutyl group or R₁ represents a C₄ to C₁₂ cycloalkyl group,
acrylates of formula CH₂═CH—COOR₂ in which R₂ represents a C₄ to C₁₂ cycloalkyl group such as isobornyl acrylate or a tert-butyl group,
(meth)acrylamides of formula: 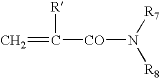
in which R₇ and R₈, which may be identical or different, each represent a hydrogen atom or a linear or branched alkyl group of 1 to 12 carbon atoms such as an n-butyl, t-butyl, isopropyl, isohexyl, isooctyl or isononyl group; or R₇ represents H and R₈ represents a 1,1-dimethyl-3-oxobutyl group, and R′ denotes H or methyl
and mixtures thereof.

35. Composition according to one of claims 32 to 34, characterized in that the monomers whose corresponding homopolymer has a glass transition temperature of greater than or equal to 40° C. are chosen from methyl methacrylate, isobutyl methacrylate and isobornyl(meth)acrylate, and mixtures thereof.

36. Composition according to one of claims 32 to 35, characterized in that the proportion of the second block with a Tg of greater than or equal to 40° C. ranges from 10% to 85%, preferably from 20% to 7-0% and better still from 30% to 70% by weight of the polymer.

37. Composition according to one of claims 27 to 31, characterized in that the second block of the block polymer has a Tg of less than or equal to 20° C. and is totally or partially derived from one or more monomers which are such that the homopolymer prepared from these monomers has a glass transition temperature of less than or equal to 20° C.

38. Composition according to one of claims 27 to 31, characterized in that the second block of the block polymer has a Tg of less than or equal to 20° C. and is a homopolymer derived from monomers which are such that the homopolymer prepared from these monomers has a glass transition temperature of less than or equal to 20° C.

39. Composition according to claim 37 or 38, characterized in that the monomers whose corresponding homopolymer has a glass transition temperature of less than or equal to 20° C. are chosen from the following monomers:
acrylates of formula CH₂═CHCOOR₃, R₃ representing a linear or branched C₁ to C₁₂ unsubstituted alkyl group, with the exemption of the tert-butyl group, in which one or more hetero atoms chosen from O, N and S is (are) optionally intercalated,
methacrylates of formula CH₂═C(CH₃)—COOR₄, R₄ representing a linear or branched C₆ to C₁₂ unsubstituted alkyl group, in which one or more hetero atoms chosen from O, N and S is (are) optionally intercalated,
vinyl esters of formula —R₅—C—O—CH═CH₂ in which R₅ represents a linear or branched C₄ to C₁₂ alkyl group,
vinyl alcohol and C₄ to C₁₂ alcohol ethers;
N—(C₄ to C₁₂)alkyl acrylamides, such as N-octylacrylamide,
and mixtures thereof.

40. Composition according to one of claims 37 to 39, characterized in that the monomers whose homopolymers have glass transition temperatures of less than or equal to 20° C. are chosen from alkyl acrylates whose alkyl chain contains from 1 to 10 carbon atoms, with the exception of the tert-butyl group.

41. Composition according to one of claims 37 to 40, characterized in that the proportion of the block with a glass transition temperature of less than or equal to 20° C. of the block polymer ranges from 20% to 90%, better still from 30% to 80% and even better still from 50% to 70% by weight of the polymer.

42. Composition according to one of the preceding claims, characterized in that the first block and/or the second block of the block polymer comprises at least one additional monomer.

43. Composition according to claim 42, characterized in that the additional monomer is chosen from hydrophilic monomers and ethylenically unsaturated monomers comprising one or more silicon atoms, and mixtures thereof.

44. Composition according to claim 42 or 43, characterized in that the additional monomer is chosen from:
ethylenically unsaturated monomers comprising at least one carboxylic or sulphonic acid function,
methacrylates of formula CH₂═C(CH₃)—COOR₆ in which R₆ represents a linear or branched alkyl group containing from 1 to 4 carbon atoms, such as a methyl, ethyl, propyl or isobutyl group, the said alkyl group being substituted with one or more substituents chosen from hydroxyl groups (for instance 2-hydroxypropyl methacrylate and 2-hydroxyethyl methacrylate) and halogen atoms (Cl, Br, I or F), such as trifluoroethyl methacrylate,
methacrylates of formula CH₂═C(CH₃)—COOR₉, R₉ representing a linear or branched C₆ to C₁₂ alkyl group in which one or more hetero atoms chosen from O, N and S is (are) optionally intercalated, the said alkyl group being substituted with one or more substituents chosen from hydroxyl groups and halogen atoms (Cl, Br, I or F);
acrylates of formula CH₂═CHCOOR₁₀, R₁₀ representing a linear or branched C₁ to C₁₂ alkyl group substituted with one or more substituents chosen from hydroxyl groups and halogen atoms (Cl, Br, I or F), such as 2-hydroxypropyl acrylate and 2-hydroxyethyl acrylate, or R₈ represents a C₁ to C₁₂ alkyl-O—POE (polyoxyethylene) with repetition of the oxyethylene unit 5 to 30 times, for example methoxy-POE, or R₁₀ represents a polyoxyethylenated group comprising from 5 to 30 ethylene oxide units,
ethylenically unsaturated monomers comprising at least one tertiary amine functional group,
and mixtures thereof.

45. Composition according to one of claims 42 to 44, characterized in that the additional monomer(s) is(are) chosen from acrylic acid, methacrylic acid, trifluoroethyl methacrylate and mixtures thereof.

46. Composition according to one of claims 42 to 45, characterized in that the additional monomer(s) represent(s) from 1 to 30% by weight of the total weight of the first and/or second blocks of the block polymer.

47. Composition according to one of the preceding claims, characterized in that each of the first and second block of the block polymer comprises at least one monomer chosen from (meth)acrylic acid esters and optionally at least one monomer chosen from (meth)acrylic acid, and mixtures thereof.

48. Composition according to one of the preceding claims, characterized in that each of the first and second block of the block polymer is totally derived from at least one monomer chosen from acrylic acid, (meth)acrylic acid esters and optionally from at least one monomer chosen from (meth)acrylic acid, and mixtures thereof.

49. Composition according to one of the preceding claims, characterized in that the difference between the glass transition temperatures (Tg) of the first and second blocks of the block polymer is greater than 10° C., better still greater than 20° C., preferably greater than 30° C. and better still greater than 40° C.

50. Composition according to one of the preceding claims, characterized in that the intermediate block of the block polymer has a glass transition temperature between the glass transition temperatures of the first and second blocks.

51. Composition according to one of the preceding claims, characterized in that the block polymer has a polydispersity index I of greater than 2, better still of greater than or equal to 2.5, preferably of greater than or equal to 2.8.

52. Composition according to one of the preceding claims, characterized in that the block polymer has a polydispersity index of between 2.8 and 6.

53. Composition according to one of the preceding claims, characterized in that the block polymer has a weight-average mass low) of less than or equal to 300 000.

54. Composition according to one of the preceding claims, characterized in that the block polymer has a weight-average mass (Mw) ranging from 35 000 to 200 000, and better still from 45.000 to 150 000.

55. Composition according to one of the preceding claims, characterized in that the block polymer has a number-average mass (Mn) of less than or equal to 70 000.

56. Composition according to one of the preceding claims, characterized in that the block polymer has a number-average mass (Mn) ranging from 10 000 to 60 000, and better still from 12 000 to 50 000.

57. Composition according to one of the preceding claims, characterized in that the block polymer is not soluble at an active material content of at least 1% by weight in water or in a mixture of water and linear or branched lower monoalcohols having from 2 to 5 carbon atoms, without modification of pH, at room temperature (25° C.).

58. Composition according to one of the preceding claims, characterized in that the block polymer is present at a dry matter (or active material) content ranging from 5 to 55% by weight, preferably ranging from 6 to 45% by weight and better still ranging from 8 to 40% by weight relative to the total weight of the composition.

59. Composition according to any one of the preceding claims, characterized in that the semicrystalline polymer is chosen from copolymers resulting from the polymerization of at least one monomer containing a crystallizable chain chosen from saturated C₁₄ to C₂₄ alkyl(meth)acrylates, C₁₁ to C₁₅ perfluoroalkyl(meth)acrylates, C₁₄ to C₂₄ N-alkyl(meth)acrylamides with or without a fluorine atom, vinyl esters containing C₁₄ to C₂₄ alkyl or perfluoroalkyl chains, vinyl ethers containing C₁₄ to C₂₄ alkyl or perfluoroalkyl chains, C₁₄ to C₂₄ alpha-olefins, para-alkylstyrenes with an alkyl group containing from 12 to 24 carbon atoms, with at least one optionally fluorinated C₁ to C₁₀ monocarboxylic acid ester or amide, which may be represented by the following formula: 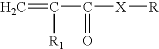
in which R₁ is H or CH₃, R represents an optionally fluorinated C₁-C₁₀ alkyl group and X represents O, NH or NR₂ in which R₂ represents an optionally fluorinated C₁-C₁₀ alkyl group.

60. Composition according to any one of the preceding claims, characterized in that the semicrystalline polymer is present in a dry matter content ranging from 0.1% to 50% by weight, preferably from 0.5% to 40% by weight, and better still from 1% to 30% by weight relative to the total weight of the composition.

61. Composition according to any one of the preceding claims, characterized in that it comprises a volatile oil.

62. Composition according to the preceding claim, characterized in that the volatile oil is chosen from hydrocarbon-based oils, silicone oils, or mixtures thereof.

63. Composition according to claim 61 or 62, characterized in that the volatile oil is present in a content ranging from 0.5% to 95% by weight, preferably from 1 to 65% by weight and better still from 5 to 40% by weight.

64. Composition according to any one of the preceding claims, characterized in that it comprises a non-volatile oil.

65. Composition according to the preceding claim, characterized in that the non-volatile oil is present in a content ranging from 0.1% to 30% by weight, preferably from 0.1% to 20% by weight, and better still from 0.1% to 10% by weight, relative to the total weight of the composition.

66. Composition according to any one of the preceding claims, characterized in that the organic liquid medium represents from 10 to 95% by weight, preferably from 20 to 90% by weight, and better still from 30 to 80% by weight, relative to the total weight of the composition.

67. Composition according to any one of the preceding claims, characterized in that it comprises an aqueous phase consisting of water or a mixture of water and a water-miscible organic solvent.

68. Composition according to the preceding claim, characterized in that the aqueous phase is present in a content ranging from 1% to 95% by weight, preferably ranging from 3% to 80% by weight, and preferably ranging from 5% to 60% by weight, relative to the total weight of the composition.

69. Composition according to any one of the preceding claims, characterized in that it comprises a wax.

70. Composition according to the preceding claim, characterized in that the total wax content of the composition ranges from 1 to 50% by weight, in particular from 5 to 30% by weight, and more particularly from 10 to 30% by weight, relative to the total weight of the composition.

71. Composition according to one of claims 1 to 68, characterized in that it is free of wax.

72. Composition according to any one of the preceding claims, characterized in that it comprises an additional film-forming polymer.

73. Composition according to any one of the preceding claims, characterized in that it comprises an additional film-forming polymer in the form of an aqueous dispersion of particles of film-forming polymer.

74. Composition according to claim 72 or 73, characterized in that the film-forming polymer is present in a dry matter content ranging from 0.1% to 60% by weight, preferably ranging from 0.5% to 40% by weight and preferably ranging from 1% to 30% by weight, relative to the total weight of the composition.

75. Composition according to any one of the preceding claims, characterized in that it comprises a surfactant.

76. Composition according to any one of the preceding claims, characterized in that it comprises an additive chosen from dyestuffs, antioxidants, fillers, pasty fatty substances, preserving agents, fragrances, neutralizers, thickeners, vitamins, coalescers and plasticizers, and mixtures thereof.

77. Composition according to one of claims 1 to 76, characterized in that it is a mascara.

78. Composition according to one of the preceding claims, characterized in that it has a dry matter content of greater than or equal to 40%, better still of greater than or equal to 45%, preferably of greater than or equal to 46%, better still of greater than or equal to 47%, even better still of greater than or equal to 48%, even more preferably of greater than or equal to 50%, better still of greater than or equal to 55%, which may be up to 60%.

79. Cosmetic process for making up or for the non-therapeutic care of keratin fibres, comprising the application to the keratin fibres of a composition according to any one of claims 1 to 78.

80. Use of a composition according to any one of the preceding claims, for obtaining makeup for the keratin fibres, in particular of the eyelashes, which is charging and/or has good resistance especially to water and/or to sweat and/or to sebum.

81. Use of the combination of a block polymer and a semicrystalline polymer in a composition for coating keratin fibres, to obtain a composition that is easy to apply to the keratin fibres and/or leading to a makeup that is charging and/or has good resistance especially to water and/or to sweat and/or to sebum on the said keratin fibres.

82. Cosmetic assembly comprising:
i) a container delimiting at least one compartment, the said container being closed by a closing member; and
ii) a composition for coating keratin fibres placed inside the said compartment, the composition being in accordance with any one of claims 1 to 78.

83. Cosmetic assembly according to claim 82, characterized in that the container consists, at least in part, of at least one thermoplastic material.

84. Cosmetic assembly according to claim 82, characterized in that the container consists, at least in part, of at least one nonthermoplastic material, especially glass or metal.

85. Assembly according to any one of claims 82 to 84, characterized in that in the closed position of the container, the closing member is screwed onto the container.

86. Assembly according to any one of claims 82 to 84, characterized in that in the closed-position of the container, the closing member is coupled to the container other than by screwing, especially by click-fastening.

87. Assembly according to any one of claims 82 to 86, characterized in that it comprises an applicator in the form of a twisted brush comprising a plurality of bristles trapped in a twisted core.

88. Assembly according to any one of claims 82 to 86, characterized in that the applicator is different from a twisted brush.
